(19) United States
(12) Patent Application Publication
(10) Pub. No.: US 2014/0275496 A1
(43) Pub. Date: Sep. 18, 2014

(54) ISOLATION OF FACTOR H FROM FRACTION I PASTE

(71) Applicants: BAXTER HEALTHCARE SA, Glattpark (Opfikon) (CH); BAXTER INTERNATIONAL INC., Deerfield, IL (US)

(72) Inventors: Shawn F. Bairstow, Gurnee, IL (US); Sindhu Ramachandran, Lake Zurich, IL (US); Richard Johnson, Mundelein, IL (US); Nicholas J. Schill, Johnsburg, IL (US)

(73) Assignees: BAXTER HEALTHCARE SA, Glattpark (Opfikon) (CH); BAXTER INTERNATIONAL INC., Deerfield, IL (US)

(21) Appl. No.: 14/213,737

(22) Filed: Mar. 14, 2014

Related U.S. Application Data
(60) Provisional application No. 61/798,212, filed on Mar. 15, 2013.

(51) Int. Cl. C07K 14/47

(52) U.S. Cl. CPC C07K 14/473
USPC 530/395

(57) ABSTRACT

Among other aspects, the present disclosure provides methods for preparing enriched compositions of plasma-derived Factor H from fractions formed during the manufacturing processes of established plasma-derived therapeutic compositions. Specifically, methods are provided for the isolation of Factor H from Fraction precipitates commonly discarded during the manufacture of commercial IgG therapeutics. Advantageously, the Factor H compositions prepared according to these methods have improved proteolytic profiles and reduced amidolytic activity.

### CROSS-REFERENCES TO RELATED APPLICATIONS

This application claims priority to U.S. Provisional Patent Application Ser. No. 61/798,212 filed Mar. 15, 2013, the disclosure of which is hereby incorporated herein by reference in its entirety for all purposes.

### BACKGROUND OF THE INVENTION

Plasma-derived therapeutic proteins, unlike other biologics that are produced via recombinant expression of DNA vectors in host cell lines, are fractionated from human blood and plasma donations. The supply of these plasma-derived products cannot be increased by simply increasing the volume of production. Rather, the level of commercially available blood products is limited by the available supply of blood and plasma donations. This dynamic results in a shortage in the availability of raw human plasma for the manufacture of plasma-derived blood factors that have lesser established commercial markets, including Factor H (FH).

Factor H is a large (155 kDa), soluble glycoprotein that functions in the regulation of the complement Alternative Pathway to ensuring that the complement system acts on pathogens and not host tissue. Factor H circulates in human plasma at a concentration of 500-800 micrograms per milliliter, binding to specific glycosaminoglycans (GAGs) present on human, but not most pathogenic, cell surfaces. Once located to self-cells and tissues, Factor H down-regulates complement activation through Factor I cofactor activity of C3b cleavage and decay accelerating activity against the alternative pathway C3 convertase, C3bBb.

Factor H is implicated as a potential therapeutic agent for several human disease states, including age-related macular degeneration (AMD), hemolytic uremic syndrome (aHUS) and membranoproliferative glomerulonephritis (MPGN). However, because of the extremely high worldwide demand for plasma-derived pooled immunoglobulin G (IgG), source plasma is not readily available for the direct isolation of Factor H. Rather, methods for Factor H isolation that can be introduced into existing IgG manufacturing schemes are needed. Several methods have been suggested to achieve just this, however, many of these proposed solutions require modification of the existing manufacturing scheme for established products. Such changes will require new regulatory approvals for the established products and may even result in alterations of the characteristics of the established products.

WO 2007/066017 describes a method for the production of Factor H preparations from cryo-poor plasma. Cryo-poor plasma, however, is a common source material for the manufacture of many commercially important IgG therapeutics, such as GAMMAGARD® LIQUID (Baxter Healthcare Corporation). WO 2007/06617 provides no guidance as to how the disclosed method, which involves passing cryo-poor plasma through at least two chromatographic steps, including anion exchange chromatography and heparin affinity chromatography, would impact, or even allow for, the manufacture IgG from the pre-processed cryo-poor plasma. In addition to requiring a complete revalidation and possible redesign of key IgG manufacturing processes, regulatory re-approval of the manufacturing procedures from key regulatory agencies would be required.

Likewise, WO 2008/113589 describes methods for the production of Factor H preparations from known plasma fractionation intermediates, namely Cohn-Oncley Fraction I supernatant, Cohn-Oncley Fraction III precipitate, and Kistler/Nitschmann Precipitate B fractions. Because these fractions are intermediates used in many commercially important IgG therapeutics, such as GAMMAGARD® LIQUID, implementation of these methods would likewise greatly impact existing IgG manufacturing capabilities.

U.S. Pat. No. 8,304,524 discloses methods for the isolation of Factor H from commonly produced by-products of IgG manufacturing processes, including Cohn Fraction I precipitate and Fraction II+III precipitate insoluble materials which are normally discarded. The '524 patent reports that about 90% of the Factor H content of plasma is fractionated into Cohn Fraction II+III precipitate during IgG manufacturing, and proposes that Factor H manufacturing efforts be focused on the extraction of Factor H from this by-product. However, Fraction II+III precipitate, and specifically the insoluble material derived therefrom and used in the '534 patent for the isolation of Factor H, contains high levels of proteolytic/amidolytic activity. It is shown herein that Factor H purified from Fraction II+III precipitate according to the methods of the '524 patent is proteolytically clipped to a large extent.

Brandstatter et al. (Vox Sanguinis (2012) 103, 201-212) report the purification of Factor H from an undisclosed plasma fraction. As in the '524 patent, Brandstatter et al. observe a large fraction of proteolytically clipped Factor H in their starting plasma fraction (see, lane 1 of the western blot shown in FIG. 3(b)).

Concerns over the amidolytic activity content of immunoglobulin compositions fueled by occurrences of thromboembolic events in patients being administered plasma-derived immunoglobulins and the recent withdrawal of two plasma-derived immunoglobulin compositions from market have highlighted a need for methods of effectively reducing serine proteases (e.g., activated protein C, kallikreins, FXIa, and FXIIa) during the manufacturing of plasma-derived therapeutics. Moreover, several studies have suggested that administration of high levels of amidolytic activity may result in unwanted thromboembolic events (Wolberg A S et al., Coagulation factor XI is a contaminant in intravenous immunoglobulin preparations. Am J Hematol 2000; 65:30-34; and Alving B M et al., Contact-activated factors: contaminants of immunoglobulin preparations with coagulant and vasoactive properties. J Lab Clin Med 1980; 96:334-346).

Fraction I is the first ethanol precipitate formed during many commercial manufacturing processes for plasma-derived IgG therapeutics. Preparation of Fraction I according to the Cohn fractionation method (Cohn et al., J. Am. Chem. Soc. 68:459-75 (1946)) results in a precipitate containing about 5.5% of the total protein content of cryo-poor plasma, the traditional starting material. The major constitute of the Fraction I precipitate is fibrinogen, representing 50-60% of the precipitated protein. Factor H, on the other hand, makes up only a small portion of the Fraction I precipitate (about 2.6% according to U.S. Pat. No. 8,304,524). As such, it is desirable to further enrich Factor H in the Fraction I precipitate prior to the use of chromatographic resins for several reasons, including reducing the amount of resin required and increasing the overall longevity of the resin.

The use of PEG for enrichment of Factor H extracted from Fraction II+III silicon dioxide filter cake was investigated in Example 7 of U.S. Pat. No. 8,304,524, the content of which is hereby expressly incorporated by reference in its entirety for all purposes and in particular for all teachings related to the use of PEG precipitation for the enrichment of Factor H precipitate extracts. In this example, PEG and alcohol precipitations of Factor H extracted from Fraction II+III silicon dioxide filter cake were compared. Observations made during these studies included that supernatants formed during ethanol precipitation contained more protein than did PEG precipitations and, accordingly, that dissolved PEG precipitates contained more proteins than did corresponding ethanol precipitates resulting in cloudier suspensions. The conclusion drawn from these experiments was that the PEG precipitates may require additional treatment prior to chromatographic methods.

Thus, a need remains for methods of manufacturing plasma-derived Factor H compositions with reduced proteolytic profiles from the existing supply of plasma donations. Advantageously, the present disclosure fulfills these and other needs by providing improved methods for purifying Factor H from Fraction I precipitates normally discarded during the manufacture of commercial plasma-derived therapeutic products.

### BRIEF SUMMARY OF INVENTION

Among other aspects, the present disclosure provides methods for preparing enriched compositions of plasma-derived Factor H from fractions formed during established IgG manufacturing processes. Specifically, methods are provided for the isolation of Factor H from Cohn Fraction I or equivalent precipitates (e.g., a Kistler-Nitschmann Fraction I) commonly discarded during the manufacture of commercial IgG therapeutics such as GAMMAGARD® LIQUID. Use of previously discarded plasma fractions eliminates the need to allocate a portion of the limited worldwide plasma resources, provided through donations, to dedicated Factor H manufacturing processes. Advantageously, it was found that Factor H compositions purified from Fraction I precipitate have lower levels of amidolytic activity and proteolytic clipping of Factor H than do compositions purified from Fraction II+III silicon dioxide filter cakes.

In some embodiments, the present disclosure provides improved methods for the purification of Factor H from Fraction I precipitates which include one or more intermediate PEG precipitations prior to chromatographic enrichment. Prior experimental work performed with Factor H compositions extracted from Fraction II+III precipitates suggested that ethanol precipitation steps could be used to enrich Factor H prior to chromatographic purification steps. However, due to fundamental differences in the alcohol concentrations used to prepare Fraction I and Fraction II+III precipitates, described in detail below, this strategy was not amenable for enrichment of Factor H from Fraction I precipitates. Advantageously, solution conditions were identified that provide substantial enrichment of Factor H extracted from Fraction I precipitates using intermediate concentrations of PEG.

In some embodiments, a method is provided for preparing an enriched Factor H composition from plasma. The method includes precipitating Factor H from a Cohn plasma pool, in a first precipitation step, the first precipitation step performed at a final concentration of 6% to 10% alcohol at a pH of 7.0 to 7.5, thereby forming a first precipitate and a first supernatant. The method also includes extracting the Factor H from the first precipitate with a first Factor H extraction buffer, thereby preparing an extracted Factor H composition. The method also includes admixing polyethylene glycol (PEG) into an extracted Factor H composition, in a second precipitation step, the second precipitation step performed at a final concentration of 2% to 7% PEG at a pH of 7.0 to 9.0, thereby forming a second precipitate and a second supernatant. The method further includes admixing PEG into the second supernatant, in a third precipitation step, the third precipitation step performed at a final concentration of 10% to 20% PEG at a pH of 7.0 to 9.0, thereby forming a third precipitate and a third supernatant. The method also includes extracting the Factor H from the third precipitate with a second Factor H extraction buffer, thereby forming an enriched Factor H composition.

In some embodiments of the methods described above, the Cohn plasma pool includes cryo-poor plasma.

In some embodiments of the methods described above, the final concentration of alcohol in the first precipitation step is 8±1%.

In some embodiments of the methods described above, the pH of the first precipitation step is 7.2±0.4.

In some embodiments of the methods described above, the first Factor H extraction buffer has a pH of from 7.0 to 9.0. In some embodiments of the methods described above, the first Factor H extraction buffer has a pH of 8.0±0.5.

In some embodiments of the methods described above, the first Factor H extraction buffer has a conductivity of 7 mS/cm to 32 mS/cm. In some embodiments of the methods described above, the first Factor H extraction buffer has a conductivity of 11 mS/cm to 22 mS/cm.

In some embodiments of the methods described above, the final concentration of PEG in the second precipitation step is 2% to 5%. In some embodiments of the methods described above, the final concentration of PEG in the second precipitation step is 4±1%.

In some embodiments of the methods described above, the pH of the second precipitation step is 8±0.5.

In some embodiments of the methods described above, the final concentration of PEG in the third precipitation step is 10% to 15%. In some embodiments of the methods described above, the final concentration of PEG in the third precipitation step is 12±1%.

In some embodiments of the methods described above, the pH of the second precipitation step is 8±0.5.

In some embodiments of the methods described above, the second Factor H extraction buffer has a pH of 7.0 to 9.0. In some embodiments of the methods described above, the second Factor H extraction buffer has a pH of 8.0±0.5.

In some embodiments of the methods described above, the second Factor H extraction buffer has a conductivity of 2 mS/cm to 10 mS/cm. In some embodiments of the methods described above, the second Factor H extraction buffer has a conductivity of 5 mS/cm to 9 mS/cm.

In some embodiments of the methods described above, the alcohol is ethanol.

In some embodiments of the methods described above, the method also includes at least one further enrichment step.

In some embodiments of the methods described above, the method also includes at least one anion exchange chromatography enrichment step. In some embodiments of the methods described above, the anion exchange chromatography enrichment step includes binding Factor H to a diethylaminoethyl (DEAE) chromatography material.

In some embodiments of the methods described above, the method also includes at least one heparin affinity chromatography enrichment step.

In some embodiments of the methods described above, the method also includes at least one size exclusion chromatography enrichment step.

In some embodiments of the methods described above, the method also includes at least one viral inactivation step.

### BRIEF DESCRIPTION OF DRAWINGS

FIG. 1 illustrates a method provided in U.S. Pat. No. 8,304,524 for purifying Factor H from Cohn Fraction II+III silicon dioxide filter cake.

FIG. 2 shows an SDS-PAGE analysis of the structure of reduced (R) and non-reduced (NR) plasma-derived Factor H (CompTech) and Factor H prepared from Fraction II+III silicon dioxide filter cake (FH002 and FH004).

FIG. 3A shows an SDS-PAGE analysis of the structure of plasma-derived Factor H (CompTech) and Factor H prepared from Fraction II+III silicon dioxide filter cake (FH006 and FH012) treated with (13 d 4° C.) and without (fresh thaw) incubation for 13 days at 4° C.

FIG. 3B shows an anti-Factor XIa western blot analysis of Factor H compositions prepared from Fraction II+III silicon dioxide filter cake (FH006 and FH012).

FIG. 4A shows an SDS-PAGE analysis of the stability of Factor H in suspended Cohn Fraction II+III silicon dioxide filter cake upon incubation at 37° C. for 24 or 48 hours in the presence or absence of protease inhibitors.

FIG. 4B shows an SDS-PAGE analysis of Factor H compositions prepared from Fraction II+III silicon dioxide filter cake (FH012, FH091410, and FH100410) with (FH100410) and without (FH012 and FH091410) the addition of protease inhibitors at the Fraction II+III extraction step.

FIG. 5 shows an anti-Factor H western blot analysis (FIG. 5A) of the structure of Factor H plasma fractions formed during the manufacture of pooled human IgG and albumin according to the fractionation scheme illustrated in FIG. 5B.

FIG. 6 illustrates the results of Factor I cofactor assays performed with plasma-derived Factor H (CompTech) and Factor H purified from Fraction II+III silicon dioxide filter cake (FH002 and FH004).

FIG. 7 illustrates the results of decay acceleration (DAF) assays performed with plasma-derived Factor H (CompTech) and Factor H purified from Fraction II+III silicon dioxide filter cake (FH002, FH004, and FH070610).

FIG. 8 illustrates the results of AH₅₀ haemolysis assays performed with plasma-derived Factor H (CompTech) and Factor H purified from Fraction II+III silicon dioxide filter cake (FH006).

FIG. 9 shows an anti-Factor H western blot analysis of the stability of Factor H in suspended Cohn Fraction I precipitate upon incubation at 37° C. for 1 hour in the presence or absence of protease inhibitors.

FIG. 10 shows SDS-PAGE (FIG. 10A) and anti-Factor H western blot (FIG. 10B) analysis of DEAE anion exchange enrichment of Factor H extracted from Cohn Fraction I precipitate.

FIG. 11 shows SDS-PAGE (FIG. 10A) and anti-Factor H western blot (FIG. 10B) analysis of heparin affinity and Q Sepharose anion exchange enrichment of Factor H extracted from Cohn Fraction I precipitate.

FIG. 12 shows an SDS-PAGE analysis of the stability of Factor H purified from Fraction I precipitate upon incubation at 4° C. for two weeks in the absence of protease inhibitors.

FIG. 13 illustrates the results of C3b ELISA assays performed with commercially available plasma-derived Factor H (CompTech), Baxter in-house recombinant Factor H (rFHlot5), Factor H purified from Fraction II+III silicon dioxide filter cake (FH12), and Factor H purified from Fraction I precipitate (189-2).

FIG. 14 shows SDS-PAGE analysis of suspended Fraction I supernatants (FIG. 14A) and precipitates (FIG. 14B) formed after addition of from 1% to 6% PEG 4 k at pH 8.0.

FIG. 15 shows anti-Factor H western blot analysis of suspended Fraction I supernatants (FIG. 14A) and precipitates (FIG. 14B) formed after addition of from 1% to 6% PEG 4 k at pH 8.0.

### DETAILED DESCRIPTION OF INVENTION

### I. Introduction

The present disclosure is based in part on the surprising discovery that Factor H compositions purified from Cohn Fraction I precipitate contain lower levels of amidolytic activity, have high percentage of intact Factor H, and generally perform better in functional assays used to evaluate the decay accelerating complement regulatory activity of Factor H. The present disclosure is also based in part on the discovery of an improved method for enriching Factor H extracted from Cohn Fraction I precipitate, which include one or more intermediate PEG precipitations.

For example, as reported in Example 3, Factor H isolated from Cohn Fraction II+III silicon dioxide filter cake is subject to extensive proteolytic clipping (compare plasma-derived Factor H in lane 7 to plasma-derived Factor H in lanes 8 and 9 of FIG. 2). In comparison, Factor H purified using the methods provided herein is largely unproteolyzed (e.g., typically 90-92% intact), and is comparable to commercially available plasma-derived Factor H preparations (compare Factor H prepared from Fraction I precipitate in lane 6 to plasma-derived Factor H in lane 9 of FIG. 12), as reported in Example 10.

Furthermore, the factor(s) responsible for Factor H proteolysis in compositions isolated from Cohn Fraction II+III silicon dioxide filter cake are co-purified with Factor H and are found in the final composition. For example, as demonstrated in Example 3, incubation of Factor H compositions purified from Fraction II+III silicon dioxide filter cake at 4° C. for 13 days results in a significant increase in proteolysis (compare freshly thawed Factor H (44% intact) in lane 3 to incubated Factor H (9% intact) in lane 2 of FIG. 3). Advantageously, Factor H purified from Fraction I precipitate according to the methods provided herein contained no detectable proteolytic clipping activity. For example, as reported in Example 10, incubation of Factor H compositions purified from Fraction I precipitate at 4° C. for two weeks resulted in no detectable increase in proteolysis (compare freshly thawed Factor H (>90% intact) in lane 6 to incubated Factor H (>90% intact) in lane 7 of FIG. 12).

Advantageously, it is shown herein that Fraction I precipitate, in contrast to Fraction II+III precipitate, contains little to none of the identified Factor H clipping activity. For example, Factor H present in Fraction I does not become proteolytically clipped when incubated in the absence of proteases inhibitors (compare fresh Fraction I precipitate in lane 5 to incubated Fraction I precipitate in lane 8 of the western blot shown in FIG. 9).

Moreover, Factor H purified according to the methods provided herein demonstrates superior activity in in vitro activities as compared to Factor H prepared from Fraction II+III silicon dioxide filter cake. For example, Factor H purified from Fraction I precipitate has C3b binding activity comparable to commercially available plasma-derived Factor H (compare Factor H purified from Fraction I precipitate (189-2) to plasma-derived Factor H(CT FH in FIG. 13), while Factor H purified from Fraction II+III silicon dioxide filter cake has greater than two-fold reduced affinity for C3b (compare Factor H purified from Fraction II+III silicon dioxide filter cake (FH12) to Factor H purified from Fraction I precipitate (189-2) in FIG. 13) in C3b ELISA experiments.

The present disclosure is also based in part on the discovery of two PEG precipitation processing steps that can be used to enrich Factor H extracted from a Fraction I precipitate prior to chromatographic purification of Factor H. Advantageously, as shown in FIGS. 14 and 15, a first set of conditions, including low PEG concentrations (e.g., 2%-6%) and slightly basic pH (e.g., 7.0-9.0), were identified that can be used to precipitate a large fraction of contaminating proteins found in the Fraction I extract without precipitating Factor H. Likewise, a second set of conditions, including moderate PEG concentrations (e.g., 10%-15%), were identified that can be used to precipitate Factor H, but not certain impurities from the Fraction I extract. These precipitation conditions can be used separately or in series to enrich Factor H extracted from Fraction I prior to chromatographic purification.

### II. DEFINITIONS

As used herein, “Factor H” refers to a protein component of the alternative pathway of complement encoded by the complement factor H gene (for example, CFH; NM000186; GeneID:3075; UniProt ID P08603; Ripoche et al., Biochem. J. 249:593-602 (1988)). Factor H is translated as a 1,213 amino acid precursor polypeptide which is processed by removal of an 18 amino acid signal peptide, resulting in the mature Factor H protein (amino acids 19-1231). As used herein, Factor H encompasses any natural variants, alternative sequences, isoforms or mutant proteins that can be found in a plasma sample, for example a human plasma sample. Examples of Factor H mutations found in the human population include, without limitation, Y402H; V62I; R78G; R127L; Δ224; Q400K; C431S; T493R; C536R; I551T; R567G; C630W; C673S; C673Y; E850K; S890I; H893R; C915S; E936D; Q950H; Y951H; T956M; C959Y; W978C; N997T; V10071; V1007L; A1010T; T1017I; Y1021F; C1043R; N1050Y; 11059T; Q1076R; R1078S; D1119G; V1134G; Y1142D; Q1143E; W1157R; C1163W; W1183L; W1183R; T1184R; L1189R; S1191L; G1194D; V1197A; E1198A; F1199S; R1210C; R1215G; R1215Q; YPTCAKR1225:1231 FQS; and P1226S. Many of the these mutations have been found to be associated with a variety of diseases and disorders, including, atypical haemolytic uremic syndrome (aHUS), age-related macular degeneration (AMD), membranoproliferative glomulonephritis type II (MPGNII), CFH deficiency, and basal laminar drusen. Factor H also includes proteins containing post-translational modifications. For example, Factor H is believed to be modified by N-acetylglucosamine (GlcNAc) at residues 529, 718, 802, 822, 882, 911, 1029, and 1095.

As used herein, the term “enriched composition” refers to a protein composition isolated from a plasma sample or cell culture supernatant, in which the purity of the protein is higher than the purity of the protein in the starting sample (e.g., pooled plasma or cell culture supernatant). In one embodiment, a protein in an enriched composition is at least 25% more pure than in the starting plasma sample. In other embodiments, an enriched composition is at least 50%, 75%, 100%, 2-fold, 3-fold, 4-fold, 5-fold, 6-fold, 7-fold, 8-fold, 9-fold, 10-fold, or more pure than the starting plasma sample. For example, an enriched Factor H composition in which 70% of the total protein is Factor H is 7-fold enriched as compared to a starting sample in which 10% of the total protein is Factor H.

As used herein, the term “cryo-poor plasma” refers to a supernatant formed by cryo-precipitation of blood plasma (e.g., plasma from a single source or a pool of plasma from multiple sources). Cryo-precipitation is typically performed by thawing frozen plasma a temperature near freezing, e.g., at a temperature below about 10° C., preferably at a temperature no higher than about 6° C. As used herein “plasma,” unless otherwise specified, refers to both recovered plasma (i.e., plasma that has been separated from whole blood ex vivo) or source plasma (i.e., plasma collected via plasmapheresis). Although cryo-precipitation is commonly performed by thawing previously frozen plasma (e.g., pooled plasma) which has already been assayed for safety and quality considerations, in some embodiments, fresh plasma may also be used. After complete thawing of the frozen plasma at low temperature, the solid cryo-precipitates are separated from the liquid supernatant (i.e., the “cryo-poor plasma”) in the cold (e.g., at a temperature below about 10° C., preferably no more than 6° C.) by centrifugation, filtration, or other suitable means.

As used herein, a “Cohn pool” or “Cohn plasma pool” refers to the starting material used for the fractionation of a plasma sample or pool of plasma samples. Cohn pools include, without limitation, whole plasma, cryo-poor plasma, and pools of whole plasma, cryo-poor plasma, or a combination thereof. In some embodiments, a Cohn pool is subjected to a pre-processing step. In certain embodiments, a Cohn pool is a cryo-poor plasma sample from which one or more blood factor have been removed in a pre-processing step, for example, adsorption onto a solid phase (e.g., aluminum hydroxide or finely divided silicon dioxide) or chromatographic step (e.g., ion exchange or heparin affinity chromatography). Various blood factors, including but not limited to, Factor Eight Inhibitor Bypass Activity (FEIBA), Factor IX-complex, Factor VII-concentrate, or Antithrombin III-complex, may be isolated from the cryo-poor plasma sample prior to use as a Cohn pool for isolation of Factor H.

As used herein, a “Cohn Fraction I precipitate” refers to a precipitate formed by the addition of from about 6% to about 10% (v/v) alcohol (e.g., denatured ethanol) at a pH of from about 7.0 to about 7.5, and encompass common intermediates formed during Cohn-Oncley (Cohn et al., J. Am. Chem. Soc. 68:459-75 (1946); Oncley et al., J. Am. Chem. Soc. 71:541-50 (1949), the disclosures of which are hereby expressly incorporated by reference in their entireties for all purposes) and Kistler-Nitschmann (Kistler and Nitschmann, Vox Sang. 7:414-424 (1962), the disclosure of which is hereby expressly incorporated by reference in its entirety for all purposes) alcohol fractionations, and derivative fractionation schemes thereof.

As used herein, the term “alcohol” refers to a C₁C₅ monohydric alcohol capable of precipitating proteins from plasma. In some embodiments, the alcohol is ethanol or methanol.

In a preferred embodiment, the alcohol is ethanol. In some embodiments, the ethanol is denatured (e.g., “denatured ethanol” or “denatured alcohol”) by addition of methanol or methyl-ethyl-keton (e.g., ethanol SDA 3A containing approximately 95% ethanol and 5% methanol (w/w)). In some embodiments, the alcohol concentrations used for precipitation reactions disclosed herein refer to a final concentration of a denatured ethanol. The skilled artisan will understand how to adapt these percentages to optimize a precipitation reaction when using a different alcohol, such as methanol.

As used herein, the term “ultrafiltration (UF)” encompasses a variety of membrane filtration methods which are typically performed in a tangential flow filtration mode. Suspended solids and solutes of high molecular weight are retained, while water and low molecular weight solutes pass through the membrane. This separation process is often used for purifying and concentrating macromolecular (10³-10⁶ Da) solutions, especially protein solutions. A number of ultrafiltration membranes are available depending on the size of the molecules they retain. Ultrafiltration is typically characterized by a membrane pore size between 1 and 1000 kDa and operating pressures between 0.01 and 10 bar.

As used herein, the term “diafiltration” is performed with the same or a similar membrane as ultrafiltration and is typically performed in a tangential flow filtration mode. During diafiltration, buffer is introduced into the recycle tank while filtrate is removed from the unit operation. In processes where the product is in the retentate (for example, Factor H), diafiltration is particularly useful for separating protein from small molecules like sugars and salts. In certain cases, diafiltration can be used to exchange the solution, buffer, or individual components of a buffering system.

As used herein, the term “about” denotes an approximate range of plus or minus 10% from a specified value. For instance, the language “about 20%” encompasses a range of 18-22%.

As used herein, the term “mixing” describes an act of causing equal distribution of two or more distinct compounds or substances in a solution or suspension by any form of agitation. Complete equal distribution of all ingredients in a solution or suspension is not required as a result of “mixing” as the term is used in this application.

As used herein, the term “solvent” encompasses any liquid substance capable of dissolving or dispersing one or more other substances. A solvent may be inorganic in nature, such as water, or it may be an organic liquid, such as ethanol, acetone, methyl acetate, ethyl acetate, hexane, petrol ether, etc. As used in the term “solvent detergent treatment,” solvent denotes an organic solvent (e.g., tri-N-butyl phosphate), which is part of the solvent detergent mixture used to inactivate lipid-enveloped viruses in solution.

As used herein, the term “detergent” is used in this application interchangeably with the term “surfactant” or “surface acting agent.” Surfactants are typically organic compounds that are amphiphilic, i.e., containing both hydrophobic groups (“tails”) and hydrophilic groups (“heads”), which render surfactants soluble in both organic solvents and water. A surfactant can be classified by the presence of formally charged groups in its head. A non-ionic surfactant has no charge groups in its head, whereas an ionic surfactant carries a net charge in its head. A zwitterionic surfactant contains a head with two oppositely charged groups. Some examples of common surfactants include: Anionic (based on sulfate, sulfonate or carboxylate anions): perfluorooctanoate (PFOA or PFO), perfluorooctanesulfonate (PFOS), sodium dodecyl sulfate (SDS), ammonium lauryl sulfate, and other alkyl sulfate salts, sodium laureth sulfate (also known as sodium lauryl ether sulfate, or SLES), alkyl benzene sulfonate; cationic (based on quaternary ammonium cations): cetyl trimethylammonium bromide (CTAB) a.k.a. hexadecyl trimethyl ammonium bromide, and other alkyltrimethylammonium salts, cetylpyridinium chloride (CPC), polyethoxylated tallow amine (POEA), benzalkonium chloride (BAC), benzethonium chloride (BZT); Long chain fatty acids and their salts: including caprylate, caprylic acid, heptanoat, hexanoic acid, heptanoic acid, nanoic acid, decanoic acid, and the like; Zwitterionic (amphoteric): dodecyl betaine; cocamidopropyl betaine; coco ampho glycinate; nonionic: alkyl poly(ethylene oxide), alkylphenol poly(ethylene oxide), copolymers of poly(ethylene oxide) and polypropylene oxide) (commercially known as Poloxamers or Poloxamines), alkyl polyglucosides, including octyl glucoside, decyl maltoside, fatty alcohols (e.g., cetyl alcohol and oleyl alcohol), cocamide MEA, cocamide DEA, polysorbates (Tween 20, Tween 80, etc.), Triton detergents, and dodecyl dimethylamine oxide.

As used herein, a “disease or disorder associated with Factor H dysfunction” refers to any disease, disorder, or condition in a subject that is caused by, characterized by, or results in a reduced level of Factor H activity in the subject. For purposes of the present invention, Factor H activity may refer to the ability of Factor H to bind a protein, protein complex, or ligand, for example, C3b, C3bBb, complement factor B (CFB), C-reactive protein, endothelial cells, glycosaminoglycans (GAGs), or alternatively, may refer to its Factor I cofactor activity or its ability to accelerate the irreversible dissociation of C3bBb. In one embodiment, a disease or disorder associated with Factor H dysfunction results in a C3 deficiency and susceptibility to bacterial infections. In some instances, diseases or disorders associated with Factor H dysfunction include conditions that are caused by or linked to mutations and polymorphism in the CFH gene encoding Factor H (for review, see, Barlow et al., Adv Exp Med Biol. 2008; 632:117-42, the disclosure of which is hereby expressly incorporated herein by reference in its entirety for all purposes). Diseases that have been linked to mutations or polymorphisms in the CFH gene include, without limitation, Factor H deficiency, atypical haemolytic uremic syndrome (aHUS), age-related macular degeneration (AMD), membranoproliferative glomulonephritis type II (MPGNII; de Cordoba and de Jorge, Clinical and Experimental Immunology 151, 1-13 (2008)), myocardial infarction (Kardys et al., Journal of the American College of Cardiology 47, 1568-1575 (2006); Mooijaart et al., Experimental Gerontology 42, 1116-1122 (2007); Nicaud et al., Journal of Molecular Medicine 85, 771-775 (2007); Pai et al., European Heart Journal 28, 1297-1303 (2007); Stark et al., Clinical Science (Lond) 113, 213-218 (2007)), coronary heart disease/coronary artery disease (CAD/CHD; (Meng et al., BMC Medical Genetics 8, 62 (2007); Pulido et al., Mayo Clinic Proceedings 82, 301-307 (2007); Topol et al., Human Molecular Genetics 15 Spec No 2, R117-R123 (2006)), and Alzheimer's disease (Hamilton et al., Neuromolecular Medicine 9, 331-334 (2007); Zetterberg et al., American Journal of Ophthalmology 143, 1059-1060 (2007)). The disclosures of the forgoing references describing the associations between mutations and polymorphisms in the CFH gene and diseases associated with Factor H dysfunction are herein incorporated by reference in their entireties for all purposes.

As used herein, a “disease or disorder associated with abnormal alternative pathway complement activity” refers to a disease, disorder, or condition that results from uncontrolled or aberrant activation of the alternative pathway of complement. Generally, uncontrolled or aberrant activation of the alternative pathway of complement can result in bystander damage of host cells and tissues, as well as a depletion of C3 and corresponding susceptibility to pathogenic infections (e.g., fungal, bacterial, viral, and protistal). Examples of diseases and disorders associated with abnormal alternative pathway complement activity include, without limitation, various autoimmune diseases (such as rheumatoid arthritis, IgA nephropathy, asthma, systemic lupus erythematosus, multiple sclerosis, Anti-Phospholipid syndrome, ANCA-associated vasculitis, pemphigus, uveitis, myathemia gravis, Hashimoto's thyroiditis), renal diseases (such as IgA nephropathy, hemolytic uremic syndrome, membranoproliferative glomerulonephritis) other disease such as asthma, Alzheimer disease, adult macular degeneration, proximal nocturnal hemoglobinuria, abdominal aortic aneurism, ischemia, and sepsis.

As used herein, the term “therapeutically effective amount or dose” or “sufficient/effective amount or dose,” refers to a dose that produces effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins; the disclosures of which are hereby expressly incorporated herein by reference in their entireties for all purposes).

As used herein, the term “pharmaceutically acceptable” means that a substance which is useful in preparing a pharmaceutical composition is generally safe, non-toxic, and neither biologically nor otherwise undesirable when administered for human pharmaceutical and/or veterinary use.

### III. PURIFICATION OF FACTOR H FROM PRECIPITATE I

In some embodiments, methods are provided for preparing an enriched Factor H composition from plasma (e.g., pooled human plasma). The method includes precipitating Factor H from plasma or a fraction thereof (e.g., cryo-poor plasma), in a first alcohol precipitation step (e.g., Fraction I precipitation) by incubating the plasma or fraction thereof after the addition of alcohol (e.g., denatured ethanol) to a final concentration of from about 6% to about 10% at a pH of from about 7.0 to about 7.5, thereby forming a first alcohol precipitate and a first alcohol supernatant. The method further includes extracting Factor H from the precipitate using a first Factor H extraction buffer, thereby forming a first Factor H composition (e.g., an extracted Factor H composition).

In some embodiments, the method further includes a step of reducing impurities from the extracted Factor H composition, by precipitating them out of solution using polyethylene glycol (PEG) in a first PEG precipitation reaction that does not substantially precipitate Factor H out of solution, thereby forming a first PEG precipitate and a first PEG supernatant. In some embodiments, the precipitation reaction is performed by incubating the extracted Factor H composition, after addition of PEG to a final concentration of from about 2% to about 7%, at a pH of from about 7.0 to about 9.0. In some embodiments, the final PEG concentration used in the precipitation reaction is from about 3% PEG to about 5% PEG. In some embodiments, the pH of the composition used in the precipitation reaction is 8.0±0.2.

In some embodiments, the method further includes a step of enriching the Factor H composition (e.g., the extracted Factor H composition or first PEG supernatant), by precipitating Factor H out of solution using polyethylene glycol (PEG) in a second PEG precipitation reaction, thereby forming a second PEG precipitate and a second PEG supernatant. In some embodiments, the precipitation reaction is performed by incubating the Factor H composition after, addition of PEG to a final concentration of at least 10%, at a pH of from 7.0 to 9.0. In some embodiments, the final PEG concentration used in the precipitation reaction is from about 10% PEG to about 15% PEG. In some embodiments, the pH of the composition used in the precipitation reaction is 8.0±0.2.

In some embodiments, in which a second PEG precipitation step is performed, the method further includes a step of extracting Factor H from the second PEG precipitate using a second Factor H extraction buffer, thereby forming an enriched Factor H composition.

In some embodiments, the Factor H composition (e.g., the first PEG supernatant, the extracted second PEG precipitate, or a derivative of either) is further enriched by one or more downstream enrichment steps, including without limitation, a further precipitation step (e.g., alcohol fractionation or polyethylene glycol fractionation), a chromatographic step (e.g., ion exchange chromatography such as anion exchange and/or cation exchange chromatography, affinity chromatography such as heparin affinity chromatography, immunoaffinity chromatography, size exclusion chromatography, or a combination thereof), a filtration step (e.g., ultrafiltration and/or diafiltration, nanofiltration, depth filtration, or sterile filtration), ultracentrifugation, electrophoretic preparation, and the like.

In some embodiments, method further includes subjecting the Factor H composition (e.g., the first PEG supernatant, the extracted second PEG precipitate, or a derivative of either) to one or more dedicated virus removal and/or inactivation steps (e.g., solvent and detergent (S/D) treatment, nanofiltration, heat treatment, or incubation at low pH).

In a specific embodiment, the method includes a first alcohol precipitation step (e.g., a Fraction I precipitation step) as described above, a first extraction step as described above, a first PEG precipitation step as described above, a second PEG precipitation step as described above, and a second extraction step as described above. In some embodiments, the method also includes one or more additional enrichment steps (e.g., anion exchange chromatography and/or heparin affinity chromatography) as described above. In some embodiments, the method also includes one or more dedicated virus removal and/or inactivation steps (e.g., S/D treatment and/or nanofiltration.

In another specific embodiment, the method includes a first alcohol precipitation step (e.g., a Fraction I precipitation step) as described above, a first extraction step as described above, and a first PEG precipitation step as described above. In some embodiments, the method also includes one or more additional enrichment steps (e.g., anion exchange chromatography and/or heparin affinity chromatography) as described above. In some embodiments, the method also includes one or more dedicated virus removal and/or inactivation steps (e.g., S/D treatment and/or nanofiltration.

In another specific embodiment, the method includes a first alcohol precipitation step (e.g., a Fraction I precipitation step) as described above, a second PEG precipitation step as described above, and a second extraction step as described above. In some embodiments, the method also includes one or more additional enrichment steps (e.g., anion exchange chromatography and/or heparin affinity chromatography) as described above. In some embodiments, the method also includes one or more dedicated virus removal and/or inactivation steps (e.g., S/D treatment and/or nanofiltration.

Further details regarding the fractionation, extraction, enrichment, and viral removal and/or activation steps described here are provided below. It is contemplated that all combinations of specific conditions (e.g., pH, temperature, precipitant concentration, and/or conductivity/ionic strength) for performing each of these individual steps can be used to perform the methods described herein for purifying a Factor H composition from Fraction I precipitate. For brevity, each of these specific conditions are not repeated here.

A. Preparation of Cryo-Poor Plasma

The starting material used for the preparation of commercial plasma-derived blood products, such as pooled IgG (e.g., IVIG or IgG for subcutaneous administration) generally consists of pooled lots of recovered plasma (i.e., plasma that has been separated from whole blood ex vivo) and/or or source plasma (i.e., plasma collected via plasmapheresis). The purification process typically starts by thawing previously frozen pooled plasma, which has already been assayed for safety and quality considerations, at a temperature no higher than 6° C. After complete thawing of the frozen plasma at low temperature, centrifugation is performed in the cold (e.g., ≦6° C.) to separate solid cryo-precipitates from the liquid supernatant. Alternatively, this separation step can be performed by filtration, rather than centrifugation. The liquid supernatant (also referred to as “cryo-poor plasma”) is then, optionally, pre-processed by removing various factors, for example factor eight inhibitor bypass activity (FEIBA), Factor IX-complex, Factor VII, antithrombin III, Prothrombin complexes, by solid phase adsorption, chromatography, etc. The final product of these steps, which is used as the starting material for the fractionation process resulting in the isolation of IgG, alpha-1-antitrypsin (A1PI), and/or albumin, is commonly referred to a the “Cohn pool.”

B. Fraction I Precipitation

To form a Fraction I precipitate (e.g., a Cohn or Kistler-Nitschmann Fraction I), the Cohn pool (e.g., cryo-poor plasma solution) is cooled to below about 6° C. (typically to about 0±1° C.) and the pH of the solution is adjusted to between about 7.0 and about 7.5. In some embodiments, the pH of the Cohn pool is adjusted to between about 7.1 and about 7.3. In a specific embodiment, the pH of the cryo-poor plasma is adjusted to a pH of at or about 7.2. Pre-cooled alcohol (typically ethanol, e.g., denatured ethanol) is then added to a target concentration of from about 6% to about 10% (v/v), typically while stirring the solution. In some embodiments, ethanol (e.g., denatured ethanol) is added to a target concentration of from about 7% to about 9% (v/v). In a specific embodiment, ethanol (e.g., denatured ethanol) is added to a target concentration of at or about 8% (v/v). At the same time the temperature is further lowered to below 0° C., typically between about −4° C. and about 0° C. In a specific embodiment, the temperature is lowered to at or about −2° C., to precipitate components of the cryo-poor plasma, including Factor H. Typically, the precipitation reaction includes a hold time of at least about 1 hour, although shorter or longer hold times may also be employed. After completion of the precipitation reaction, the supernatant (e.g., “Supernatant I”) is then separated from the precipitate (e.g., “Fraction I” precipitate) by centrifugation, filtration, or other suitable means.

Fraction I precipitation was previously used to remove fibrinogen and other impurities from plasma during the manufacturing therapeutic compositions such as pooled IgG (e.g., IVIG or IgG for subcutaneous administration). Thus, the resulting Fraction I precipitate is commonly discarded during industrial fractionation of pooled plasma. Advantageously, it was found that a significant fraction of Factor H (e.g., about 10% of the total content found in plasma) is present in this precipitate. Furthermore, it is shown herein that Factor H compositions purified from Fraction I precipitate have lower levels or amidolytic activity, higher contents of intact Factor H, and higher biological activities (e.g., higher specific activity is AH₅₀ heamolysis activity assays and higher affinity for surface bound C3b) than do Factor H compositions purified from other Factor H containing fractions (e.g., Fraction II+III precipitates and derivatives thereof). Accordingly, methods for purifying Factor H from Fraction I precipitate are provided herein. Suitable buffers and methods for the extraction and enrichment of Factor H from Factor I precipitates are provided below.

As compared to conventional methods for Fraction I precipitation, (e.g., as described in Cohn et al., supra; Oncley et al., supra), some embodiments provided herein result in improved yields of plasma factors (e.g., Factor H). In one embodiment, the precipitating alcohol is added in a fashion that finely disperses or that rapidly disperses the alcohol at the point of addition. For example, in one embodiment, precipitating alcohol is added to the plasma or derivative thereof (e.g., cryo-poor plasma) by spraying. In a second embodiment, precipitating alcohol is added to the plasma or derivative thereof (e.g., cryo-poor plasma) from below or directly adjacent to a stirring apparatus (e.g., an impeller). Addition of alcohol by any of these mechanisms avoids local over-concentration of alcohol which occurs, for example, at the point of fluent addition and results in the irreversible denaturation of proteins and/or precipitation of proteins that would otherwise be recovered in the supernatant.

In another embodiment, one or more pH modifying agent is added in a fashion that finely disperses or that rapidly disperses the pH modifying agent at the point of addition. For example, in one embodiment, the pH modifying agent is added by spraying. In a second embodiment, the pH modifying agent is added from below or directly adjacent to a stirring apparatus (e.g., an impeller). In a third embodiment, the pH modifying agent is added by sprinkling a solid form of the agent over a delocalized area.

In some embodiments, the pH of the solution is adjusted after addition of the precipitating alcohol. In some embodiments, the pH of the solution is adjusted during the addition of the precipitating alcohol. In some embodiments, the pH of the solution is adjusted in any combination of prior to, during, and after addition of the precipitating alcohol. In some embodiments, the pH of the solution is maintained at the desired pH throughout the precipitation incubation by monitoring and adjusting the pH of the solution as needed. In a preferred embodiment, the alcohol is ethanol (e.g., denatured ethanol).

Although the process for preparing a Fraction I precipitate is described above as a linear process, the skilled artisan will understand that the order of individual steps may be switched, combined, and/or reordered. For example, in some implementations, the pH of the Cohn pool may be adjusted prior to, during, and/or after cooling the solution down to a target temperature.

C. Extraction of Factor H from Fraction I Precipitate

To extract Factor H from the Fraction I precipitate, an extraction buffer is used to suspend the Fractionation I precipitate. In some embodiments, suspension includes addition of the extraction buffer to the Fraction I precipitate followed by stirring (e.g., by hand, with stir bar, or with an impeller). In some embodiments, extracting Factor H includes mechanically breaking down the Fraction I precipitate, before or after addition of the extraction buffer. Examples of techniques that can be used to break down the Fraction I precipitate include, without limitation, cutting the precipitate into small pieces (e.g., with one or more blades), mashing the precipitate, blending the precipitate, grinding the precipitate, and using pressure or sonication to homogenize the precipitate. Methods for implementing these strategies are known in the art.

In some embodiments, the Fraction I precipitate is suspended at a ratio of 1 part precipitate to from about 4 parts to about 40 parts extraction buffer. Other suitable ranges for the suspension ratio include, without limitation, from about 1:8 to about 1:30, from about 1:10 to about 1:20, from about 1:12 to about 1:18, from about 1:13 to about 1:17, and from about 1:14 to about 1:16. Some embodiments, suspension ratio is about 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, or higher. In a specific embodiment, the suspension ratio is about 1 part precipitate to about 15 parts suspension buffer. In another specific embodiment, the suspension ratio is about 1 part precipitate to about 20 parts suspension buffer.

In some embodiments, the Factor H extraction buffer has a pH between about 4.0 and about 10. In some embodiments, the Factor H extraction buffer has a pH of 5.0±1, 5.5±1, 6.0±1, 6.5±1, 7.0±1, 7.5±1, 8.0±1, 8.5±1, 9.0±1, 6.0±2, 6.5±2, 7.0±2, 7.5±2, or 8.0±2. In some embodiments, the Factor H extraction buffer has a pH above or below (e.g., by at least 0.3 pH units) the isoelectric point of Factor H, determined to be 6.0 (Nagaki et al, 1978; Pio et al, 2001; and Brandstatter et al, (2012)). In some embodiments, the pH of the extraction buffer is from about 6.5 to about 10. In some embodiments, the Factor H extraction buffer has a pH of 7.0±0.5, 7.5±0.5, 8.0±0.5, 8.5±0.5, 9.0±0.5, 9.5±0.5. In some embodiments, the pH of the extraction buffer is from about 4.0 to about 5.5. In some embodiments, the Factor H extraction buffer has a pH of 4.5±0.5 or 5.0±0.5.

Generally, these pH requirements can be met using a buffering agent, including without limitation, acetate, citrate, monobasic phosphate, dibasic phosphate, mixtures thereof, and the like. Suitable buffer concentrations typically range from about 2.5 mM to about 100 mM, or from about 5 mM to about 50 mM, or about 2.5 mM, 5 mM, 10 mM, 15 mM, 20 mM, 25 mM, 50 mM, 75 mM, 100 mM, 125 mM, 150 mM, 175 mM, or 200 mM buffering agent.

In some embodiments, the extraction buffer has a conductivity equal to that of a solution of from about 50 to about 500 mM sodium chloride. In some embodiments, the extraction buffer has a conductivity equal to that of a solution of from about 100 to about 300 mM sodium chloride. In a specific embodiment, the extraction buffer has a conductivity equal to that of a solution of from about 150 to about 200 mM sodium chloride.

In some embodiments, the extraction buffer includes from 10 to 100 mM buffering agent (e.g., Tris), from 150 to 250 mM of an alkaline metal chloride salt (e.g., sodium chloride), optionally, from 1 to 10 mM of a metal chelating agent (e.g., EDTA and/or EGTA), and a pH from 7.5 to 8.5. In a specific embodiment, the extraction buffer includes 20 to 40 mM Tris, 175 to 225 mM sodium chloride, 5±1 mM EDTA, and a pH of 8.0±0.2.

Generally, the extraction is performed at a temperature of from about 0° C. and about 25° C. In certain embodiments, the extraction is performed at about 0° C., 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., or 25° C. In a particular embodiment, the extraction is performed at around room temperature (about 20° C.). In another particular embodiment, the extraction is performed at from about 2° C. to about 10° C. In some embodiments, the extraction process is performed under continuous agitation (e.g., stirring, cutting, mashing, blending, grinding, or otherwise homogenizing) until all soluble components of the Fraction I precipitate are brought into solution. In certain embodiments, the extraction will proceed for at or about between 30 and 300 minutes, or for at or between 120 and 240 min, or for at or about between 150 and 210 minutes. In certain embodiments, the extraction process will proceed for about 30, 45, 60, 90, 120, 150, 180, 210, 240, 270, 300, or more minutes, optionally, with continuous agitation.

D. First PEG Precipitation

In some embodiments, the method further includes a step of precipitating impurities from the Fraction I extract using low concentrations of PEG under conditions in which Factor H is substantially not co-precipitated. The resulting PEG precipitate and supernatant are referred to herein as the “first PEG precipitate” and “first PEG supernatant,” respectively. In some embodiments, this step includes precipitating at least one impurity (e.g., a lipid or non-Factor H protein) from the composition and then separating the resulting precipitate from the supernatant containing Factor H.

In some embodiments, the first PEG precipitation step is accomplished by incubating the Fraction I extract, after addition of PEG (e.g., PEG 4 k) to a final concentration of from about 2% to about 7%, at a pH of from about 7.0 to about 9.0. In some embodiments, precipitation of a particular impurity of interest may be facilitated by matching, or nearly matching, the pH of the precipitation reaction to the isoelectric point of the impurity, i.e., isoelectric point precipitation.

In some embodiments, the concentration of PEG used in the reaction is adjusted to maximize the precipitation of one or more impurities and/or to minimize the precipitation of Factor H. In some embodiments, the first PEG precipitation step is performed using a final PEG concentration of from about 3% to about 6% or from about 4% to about 5%. In certain embodiments, the final PEG concentration of the precipitation is about 2%, 3%, 4%, 5%, 6%, or 7%.

Generally, the nomenclature used to refer to a particular preparation of PEG includes the average molecular weight of PEG in the preparation. For example, “PEG 4 k” or “PEG 4000” refers to a preparation of PEG having an average molecular weight of about 4 kDa. Depending on the grade of PEG used, individual PEG preparations contain a distribution of average molecular weights. In accordance with the methods provided herein, PEG of any average molecular weight and distribution of molecular weights may be used for the precipitation of impurities from the Fraction I extract. Non-limiting examples of PEGs useful for the methods described herein include PEG lk, PEG 3350, PEG 4 k, PEG 10 k, PEG 12 k, PEG 15 k, PEG 20 k, PEG 30 k, and PEG 40 k. In a particular embodiment, the PEG is PEG 4 k.

In some embodiments, the pH of the reaction is adjusted to maximize the precipitation of one or more impurities and/or to minimize the precipitation of Factor H. In certain embodiments, the pH of the solution is adjusted to about 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, or 9.0. In one embodiment, the pH of the solution is adjusted to from about 7.2 to about 8.8. In another embodiment, the pH of the solution is adjusted to from about 7.3 to about 8.7. In another embodiment, the pH of the solution is adjusted to from about 7.4 to about 8.6. In another embodiment, the pH of the solution is adjusted to from about 7.5 to about 8.5. In another embodiment, the pH of the solution is adjusted to from about 7.6 to about 8.4. In another embodiment, the pH of the solution is adjusted from about 7.7 to about 8.3. In another embodiment, the pH of the solution is adjusted to from about 7.8 to about 8.2. In another embodiment, the pH of the solution is adjusted to from about 7.9 to about 8.1. In another embodiment, the pH of the solution is adjusted to at or about 8.0.

In one embodiment, the first PEG precipitation is performed using a final concentration of about 2% to about 7% PEG at a pH of from about 7.0 to about 9.0. In one embodiment, the first PEG precipitation is performed using a final concentration of about 2% to about 7% PEG at a pH of from about 7.5 to about 8.5. In one embodiment, the first PEG precipitation is performed using a final concentration of about 2% to about 7% PEG at a pH of 8.0±0.2. In one embodiment, the first PEG precipitation is performed using a final concentration of about 3% to about 6% PEG at a pH of from about 7.0 to about 9.0. In one embodiment, the first PEG precipitation is performed using a final concentration of about 3% to about 6% PEG at a pH of from about 7.5 to about 8.5. In one embodiment, the first PEG precipitation is performed using a final concentration of about 3% to about 6% PEG at a pH of 8.0±0.2. In one embodiment, the first PEG precipitation is performed using a final concentration of 4.5±1.0% PEG at a pH of from about 7.0 to about 9.0. In one embodiment, the first PEG precipitation is performed using a final concentration of 4.5±1.0% PEG at a pH of from about 7.5 to about 8.5. In one embodiment, the first PEG precipitation is performed using a final concentration of 4.5±1.0% PEG at a pH of 8.0±0.2. In one embodiment, the first PEG precipitation is performed using a final concentration of 4.5±0.5% PEG at a pH of from about 7.0 to about 9.0. In one embodiment, the first PEG precipitation is performed using a final concentration of 4.5±0.5% PEG at a pH of from about 7.5 to about 8.5. In one embodiment, the first PEG precipitation is performed using a final concentration of 4.5±0.5% PEG at a pH of 8.0±0.2. In one embodiment, the first PEG precipitation is performed using a final PEG concentration and pH combination selected from variations 1 to 850 in Table 1.

TABLE 1
Useful combinations of PEG concentration and pH for the first PEG precipitation step.

| | Final PEG Percentage | | | | | | | | | | | | | | | | |
|---|---|---|---|---|---|---|---|---|---|---|---|---|---|---|---|---|---|
| | 4.5 ± | 4.5 ± | 4.5 ± | 4.5 ± | 4.5 ± | | | | | | | | | | | | |
| pH | 0.5% | 1.0% | 1.5% | 2.0% | 2.5% | 3 ± 1% | 4 ± 1% | 5 ± 1% | 6 ± 1% | 4 ± 2% | 5 ± 2% | 2.0 | 3.0 | 4.0 | 5.0 | 6.0 | 7.0 |
| 8.0 ± 1.0 | Var 1 | Var | Var | Var | Var | Var | Var | Var | Var | Var | Var | Var | Var | Var | Var | Var | Var |
| | | 51 | 101 | 151 | 201 | 251 | 301 | 351 | 401 | 451 | 501 | 551 | 601 | 651 | 701 | 751 | 801 |
| 8.0 ± 0.9 | Var 2 | Var | Var | Var | Var | Var | Var | Var | Var | Var | Var | Var | Var | Var | Var | Var | Var |
| | | 52 | 102 | 152 | 202 | 252 | 302 | 352 | 402 | 452 | 502 | 552 | 602 | 652 | 702 | 752 | 802 |
| 8.0 ± 0.8 | Var 3 | Var | Var | Var | Var | Var | Var | Var | Var | Var | Var | Var | Var | Var | Var | Var | Var |
| | | 53 | 103 | 153 | 203 | 253 | 303 | 353 | 403 | 453 | 503 | 553 | 603 | 653 | 703 | 753 | 803 |
| 8.0 ± 0.7 | Var 4 | Var | Var | Var | Var | Var | Var | Var | Var | Var | Var | Var | Var | Var | Var | Var | Var |
| | | 54 | 104 | 154 | 204 | 254 | 304 | 354 | 404 | 454 | 504 | 554 | 604 | 654 | 704 | 754 | 804 |
| 8.0 ± 0.6 | Var 5 | Var | Var | Var | Var | Var | Var | Var | Var | Var | Var | Var | Var | Var | Var | Var | Var |
| | | 55 | 105 | 155 | 205 | 255 | 305 | 355 | 405 | 455 | 505 | 555 | 605 | 655 | 705 | 755 | 805 |
| 8.0 ± 0.5 | Var 6 | Var | Var | Var | Var | Var | Var | Var | Var | Var | Var | Var | Var | Var | Var | Var | Var |
| | | 56 | 106 | 156 | 206 | 256 | 306 | 356 | 406 | 456 | 506 | 556 | 606 | 656 | 706 | 756 | 806 |
| 8.0 ± 0.4 | Var 7 | Var | Var | Var | Var | Var | Var | Var | Var | Var | Var | Var | Var | Var | Var | Var | Var |
| | | 57 | 107 | 157 | 207 | 257 | 307 | 357 | 407 | 457 | 507 | 557 | 607 | 657 | 707 | 757 | 807 |
| 8.0 ± 0.3 | Var 8 | Var | Var | Var | Var | Var | Var | Var | Var | Var | Var | Var | Var | Var | Var | Var | Var |
| | | 58 | 108 | 158 | 208 | 258 | 308 | 358 | 408 | 458 | 508 | 558 | 608 | 658 | 708 | 758 | 808 |
| 8.0 ± 0.2 | Var 9 | Var | Var | Var | Var | Var | Var | Var | Var | Var | Var | Var | Var | Var | Var | Var | Var |
| | | 59 | 109 | 159 | 209 | 259 | 309 | 359 | 409 | 459 | 509 | 559 | 609 | 659 | 709 | 759 | 809 |
| 8.0 ± 0.1 | Var | Var | Var | Var | Var | Var | Var | Var | Var | Var | Var | Var | Var | Var | Var | Var | Var |
| | 10 | 60 | 110 | 160 | 210 | 260 | 310 | 360 | 410 | 460 | 510 | 560 | 610 | 660 | 710 | 760 | 810 |
| 8.0 | Var | Var | Var | Var | Var | Var | Var | Var | Var | Var | Var | Var | Var | Var | Var | Var | Var |
| | 11 | 61 | 111 | 161 | 211 | 261 | 311 | 361 | 411 | 461 | 511 | 561 | 611 | 661 | 711 | 761 | 811 |
| 7.0 ± 0.2 | Var | Var | Var | Var | Var | Var | Var | Var | Var | Var | Var | Var | Var | Var | Var | Var | Var |
| | 12 | 62 | 112 | 162 | 212 | 262 | 312 | 362 | 412 | 462 | 512 | 562 | 612 | 662 | 712 | 762 | 812 |
| 7.1 ± 0.2 | Var | Var | Var | Var | Var | Var | Var | Var | Var | Var | Var | Var | Var | Var | Var | Var | Var |
| | 13 | 63 | 113 | 163 | 213 | 263 | 313 | 363 | 413 | 463 | 513 | 563 | 613 | 663 | 713 | 763 | 813 |
| 7.2 ± 0.2 | Var | Var | Var | Var | Var | Var | Var | Var | Var | Var | Var | Var | Var | Var | Var | Var | Var |
| | 14 | 64 | 114 | 164 | 214 | 264 | 314 | 364 | 414 | 464 | 514 | 564 | 614 | 664 | 714 | 764 | 814 |
| 7.3 ± 0.2 | Var | Var | Var | Var | Var | Var | Var | Var | Var | Var | Var | Var | Var | Var | Var | Var | Var |
| | 15 | 65 | 115 | 165 | 215 | 265 | 315 | 365 | 415 | 465 | 515 | 565 | 615 | 665 | 715 | 765 | 815 |
| 7.4 ± 0.2 | Var | Var | Var | Var | Var | Var | Var | Var | Var | Var | Var | Var | Var | Var | Var | Var | Var |
| | 16 | 66 | 116 | 166 | 216 | 266 | 316 | 366 | 416 | 466 | 516 | 566 | 616 | 666 | 716 | 766 | 816 |
| 7.5 ± 0.2 | Var | Var | Var | Var | Var | Var | Var | Var | Var | Var | Var | Var | Var | Var | Var | Var | Var |
| | 17 | 67 | 117 | 167 | 217 | 267 | 317 | 367 | 417 | 467 | 517 | 567 | 617 | 667 | 717 | 767 | 817 |
| 7.6 ± 0.2 | Var | Var | Var | Var | Var | Var | Var | Var | Var | Var | Var | Var | Var | Var | Var | Var | Var |
| | 18 | 68 | 118 | 168 | 218 | 268 | 318 | 368 | 418 | 468 | 518 | 568 | 618 | 668 | 718 | 768 | 818 |
| 7.7 ± 0.2 | Var | Var | Var | Var | Var | Var | Var | Var | Var | Var | Var | Var | Var | Var | Var | Var | Var |
| | 19 | 69 | 119 | 169 | 219 | 269 | 319 | 369 | 419 | 469 | 519 | 569 | 619 | 669 | 719 | 769 | 819 |
| 7.8 ± 0.2 | Var | Var | Var | Var | Var | Var | Var | Var | Var | Var | Var | Var | Var | Var | Var | Var | Var |
| | 20 | 70 | 120 | 170 | 220 | 270 | 320 | 370 | 420 | 470 | 520 | 570 | 620 | 670 | 720 | 770 | 820 |
| 7.9 ± 0.2 | Var | Var | Var | Var | Var | Var | Var | Var | Var | Var | Var | Var | Var | Var | Var | Var | Var |
| | 21 | 71 | 121 | 171 | 221 | 271 | 321 | 371 | 421 | 471 | 521 | 571 | 621 | 671 | 721 | 771 | 821 |
| 8.1 ± 0.2 | Var | Var | Var | Var | Var | Var | Var | Var | Var | Var | Var | Var | Var | Var | Var | Var | Var |
| | 22 | 72 | 122 | 172 | 222 | 272 | 322 | 372 | 422 | 472 | 522 | 572 | 622 | 672 | 722 | 772 | 822 |
| 8.2 ± 0.2 | Var | Var | Var | Var | Var | Var | Var | Var | Var | Var | Var | Var | Var | Var | Var | Var | Var |
| | 23 | 73 | 123 | 173 | 223 | 273 | 323 | 373 | 423 | 473 | 523 | 573 | 623 | 673 | 723 | 773 | 823 |
| 8.3 ± 0.2 | Var | Var | Var | Var | Var | Var | Var | Var | Var | Var | Var | Var | Var | Var | Var | Var | Var |
| | 24 | 74 | 124 | 174 | 224 | 274 | 324 | 374 | 424 | 474 | 524 | 574 | 624 | 674 | 724 | 774 | 824 |
| 8.4 ± 0.2 | Var | Var | Var | Var | Var | Var | Var | Var | Var | Var | Var | Var | Var | Var | Var | Var | Var |
| | 25 | 75 | 125 | 175 | 225 | 275 | 325 | 375 | 425 | 475 | 525 | 575 | 625 | 675 | 725 | 775 | 825 |
| 8.5 ± 0.2 | Var | Var | Var | Var | Var | Var | Var | Var | Var | Var | Var | Var | Var | Var | Var | Var | Var |
| | 26 | 76 | 126 | 176 | 226 | 276 | 326 | 376 | 426 | 476 | 526 | 576 | 626 | 676 | 726 | 776 | 826 |
| 8.6 ± 0.2 | Var | Var | Var | Var | Var | Var | Var | Var | Var | Var | Var | Var | Var | Var | Var | Var | Var |
| | 27 | 77 | 127 | 177 | 227 | 277 | 327 | 377 | 427 | 477 | 527 | 577 | 627 | 677 | 727 | 777 | 827 |
| 8.7 ± 0.2 | Var | Var | Var | Var | Var | Var | Var | Var | Var | Var | Var | Var | Var | Var | Var | Var | Var |
| | 28 | 78 | 128 | 178 | 228 | 278 | 328 | 378 | 428 | 478 | 528 | 578 | 628 | 678 | 728 | 778 | 828 |
| 8.8 ± 0.2 | Var | Var | Var | Var | Var | Var | Var | Var | Var | Var | Var | Var | Var | Var | Var | Var | Var |
| | 29 | 79 | 129 | 179 | 229 | 279 | 329 | 379 | 429 | 479 | 529 | 579 | 629 | 679 | 729 | 779 | 829 |
| 8.9 ± 0.2 | Var | Var | Var | Var | Var | Var | Var | Var | Var | Var | Var | Var | Var | Var | Var | Var | Var |
| | 30 | 80 | 130 | 180 | 230 | 280 | 330 | 380 | 430 | 480 | 530 | 580 | 630 | 680 | 730 | 780 | 830 |
| 9.0 ± 0.2 | Var | Var | Var | Var | Var | Var | Var | Var | Var | Var | Var | Var | Var | Var | Var | Var | Var |
| | 31 | 81 | 131 | 181 | 231 | 281 | 331 | 381 | 431 | 481 | 531 | 581 | 631 | 681 | 731 | 781 | 831 |
| 7.1 ± 0.1 | Var | Var | Var | Var | Var | Var | Var | Var | Var | Var | Var | Var | Var | Var | Var | Var | Var |
| | 32 | 82 | 132 | 182 | 232 | 282 | 332 | 382 | 432 | 482 | 532 | 582 | 632 | 682 | 732 | 782 | 832 |
| 7.2 ± 0.1 | Var | Var | Var | Var | Var | Var | Var | Var | Var | Var | Var | Var | Var | Var | Var | Var | Var |
| | 33 | 83 | 133 | 183 | 233 | 283 | 333 | 383 | 433 | 483 | 533 | 583 | 633 | 683 | 733 | 783 | 833 |
| 7.3 ± 0.1 | Var | Var | Var | Var | Var | Var | Var | Var | Var | Var | Var | Var | Var | Var | Var | Var | Var |
| | 34 | 84 | 134 | 184 | 234 | 284 | 334 | 384 | 434 | 484 | 534 | 584 | 634 | 684 | 734 | 784 | 834 |
| 7.4 ± 0.1 | Var | Var | Var | Var | Var | Var | Var | Var | Var | Var | Var | Var | Var | Var | Var | Var | Var |
| | 35 | 85 | 135 | 185 | 235 | 285 | 335 | 385 | 435 | 485 | 535 | 585 | 635 | 685 | 735 | 785 | 835 |
| 7.5 ± 0.1 | Var | Var | Var | Var | Var | Var | Var | Var | Var | Var | Var | Var | Var | Var | Var | Var | Var |
| | 36 | 86 | 136 | 186 | 236 | 286 | 336 | 386 | 436 | 486 | 536 | 586 | 636 | 686 | 736 | 786 | 836 |
| 7.6 ± 0.1 | Var | Var | Var | Var | Var | Var | Var | Var | Var | Var | Var | Var | Var | Var | Var | Var | Var |
| | 37 | 87 | 137 | 187 | 237 | 287 | 337 | 387 | 437 | 487 | 537 | 587 | 637 | 687 | 737 | 787 | 837 |
| 7.7 ± 0.1 | Var | Var | Var | Var | Var | Var | Var | Var | Var | Var | Var | Var | Var | Var | Var | Var | Var |
| | 38 | 88 | 138 | 188 | 238 | 288 | 338 | 388 | 438 | 488 | 538 | 588 | 638 | 688 | 738 | 788 | 838 |
| 7.8 ± 0.1 | Var | Var | Var | Var | Var | Var | Var | Var | Var | Var | Var | Var | Var | Var | Var | Var | Var |
| | 39 | 89 | 139 | 189 | 239 | 289 | 339 | 389 | 439 | 489 | 539 | 589 | 639 | 689 | 739 | 789 | 839 |
| 7.9 ± 0.1 | Var | Var | Var | Var | Var | Var | Var | Var | Var | Var | Var | Var | Var | Var | Var | Var | Var |
| | 40 | 90 | 140 | 190 | 240 | 290 | 340 | 390 | 440 | 490 | 540 | 590 | 640 | 690 | 740 | 790 | 840 |
| 8.1 ± 0.1 | Var | Var | Var | Var | Var | Var | Var | Var | Var | Var | Var | Var | Var | Var | Var | Var | Var |
| | 41 | 91 | 141 | 191 | 241 | 291 | 341 | 391 | 441 | 491 | 541 | 591 | 641 | 691 | 741 | 791 | 841 |
| 8.2 ± 0.1 | Var | Var | Var | Var | Var | Var | Var | Var | Var | Var | Var | Var | Var | Var | Var | Var | Var |
| | 42 | 92 | 142 | 192 | 242 | 292 | 342 | 392 | 442 | 492 | 542 | 592 | 642 | 692 | 742 | 792 | 842 |
| 8.3 ± 0.1 | Var | Var | Var | Var | Var | Var | Var | Var | Var | Var | Var | Var | Var | Var | Var | Var | Var |
| | 43 | 93 | 143 | 193 | 243 | 293 | 343 | 393 | 443 | 493 | 543 | 593 | 643 | 693 | 743 | 793 | 843 |
| 8.4 ± 0.1 | Var | Var | Var | Var | Var | Var | Var | Var | Var | Var | Var | Var | Var | Var | Var | Var | Var |
| | 44 | 94 | 144 | 194 | 244 | 294 | 344 | 394 | 444 | 494 | 544 | 594 | 644 | 694 | 744 | 794 | 844 |
| 8.5 ± 0.1 | Var | Var | Var | Var | Var | Var | Var | Var | Var | Var | Var | Var | Var | Var | Var | Var | Var |
| | 45 | 95 | 145 | 195 | 245 | 295 | 345 | 395 | 445 | 495 | 545 | 595 | 645 | 695 | 745 | 795 | 845 |
| 8.6 ± 0.1 | Var | Var | Var | Var | Var | Var | Var | Var | Var | Var | Var | Var | Var | Var | Var | Var | Var |
| | 46 | 96 | 146 | 196 | 246 | 296 | 346 | 396 | 446 | 496 | 546 | 596 | 646 | 696 | 746 | 796 | 846 |
| 8.7 ± 0.1 | Var | Var | Var | Var | Var | Var | Var | Var | Var | Var | Var | Var | Var | Var | Var | Var | Var |
| | 47 | 97 | 147 | 197 | 247 | 297 | 347 | 397 | 447 | 497 | 547 | 597 | 647 | 697 | 747 | 797 | 847 |
| 8.8 ± 0.1 | Var | Var | Var | Var | Var | Var | Var | Var | Var | Var | Var | Var | Var | Var | Var | Var | Var |
| | 48 | 98 | 148 | 198 | 248 | 298 | 348 | 398 | 448 | 498 | 548 | 598 | 648 | 698 | 748 | 798 | 848 |
| 8.9 ± 0.1 | Var | Var | Var | Var | Var | Var | Var | Var | Var | Var | Var | Var | Var | Var | Var | Var | Var |
| | 49 | 99 | 149 | 199 | 249 | 299 | 349 | 399 | 449 | 499 | 549 | 599 | 649 | 699 | 749 | 799 | 849 |
| 9.0 ± 0.1 | Var | Var | Var | Var | Var | Var | Var | Var | Var | Var | Var | Var | Var | Var | Var | Var | Var |
| | 50 | 100 | 150 | 200 | 250 | 300 | 350 | 400 | 450 | 500 | 550 | 600 | 650 | 700 | 750 | 800 | 850 |

Var = Variation

In one embodiment, the first PEG precipitation step is performed using PEG having an average molecular weight of from 1 kD to 20 kD and a final concentration and pH combination selected from variations 1 to 850 in Table 1. In one embodiment, the first PEG precipitation step is performed using PEG having an average molecular weight of from 1 kD to 10 kD and a final concentration and pH combination selected from variations 1 to 850 in Table 1. In one embodiment, the first PEG precipitation step is performed using PEG having an average molecular weight of from 1 kD to 7.5 kD and a final concentration and pH combination selected from variations 1 to 850 in Table 1. In one embodiment, the first PEG precipitation step is performed using PEG having an average molecular weight of from 2 kD to 20 kD and a final concentration and pH combination selected from variations 1 to 850 in Table 1. In one embodiment, the first PEG precipitation step is performed using PEG having an average molecular weight of from 2 kD to 20 kD and a final concentration and pH combination selected from variations 1 to 850 in Table 1. In one embodiment, the first PEG precipitation step is performed using PEG having an average molecular weight of from 2 kD to 10 kD and a final concentration and pH combination selected from variations 1 to 850 in Table 1. In one embodiment, the first PEG precipitation step is performed using PEG having an average molecular weight of from 2 kD to 7.5 kD and a final concentration and pH combination selected from variations 1 to 850 in Table 1. In one embodiment, the first PEG precipitation step is performed using PEG having an average molecular weight of from 2 kD to 5 kD and a final concentration and pH combination selected from variations 1 to 850 in Table 1. In one embodiment, the first PEG precipitation step is performed using PEG having an average molecular weight of from 3.35 kD to 20 kD and a final concentration and pH combination selected from variations 1 to 850 in Table 1. In one embodiment, the first PEG precipitation step is performed using PEG having an average molecular weight of from 3.35 kD to 10 kD and a final concentration and pH combination selected from variations 1 to 850 in Table 1. In one embodiment, the first PEG precipitation step is performed using PEG having an average molecular weight of from 3.35 kD to 7.5 kD and a final concentration and pH combination selected from variations 1 to 850 in Table 1. In one embodiment, the first PEG precipitation step is performed using PEG having an average molecular weight of from 3.35 kD to 5 kD and a final concentration and pH combination selected from variations 1 to 850 in Table 1. In one embodiment, the first PEG precipitation step is performed using PEG having an average molecular weight of 4±1 kD and a final concentration and pH combination selected from variations 1 to 850 in Table 1. In one embodiment, the first PEG precipitation step is performed using PEG having an average molecular weight of 4±2 kD and a final concentration and pH combination selected from variations 1 to 850 in Table 1.

Depending on factors including, but not limited to, the particular PEG used, a desired concentration of PEG, a desired pH for the reaction, and a desired precipitation profile, one of skill in the art will readily be able to modify the precipitation reactions described herein to achieve a desired result (e.g., balancing maximal precipitation of targeted impurities with minimal precipitation of Factor H).

E. Second PEG Precipitation

In some embodiments, the method further includes a step of precipitating Factor H from a Factor H composition (e.g., a Fraction I extract or first PEG supernatant) using high concentrations of PEG under conditions in which Factor H is substantially precipitated. The resulting PEG precipitate and supernatant are referred to herein as the “second PEG precipitate” and “second PEG supernatant,” respectively, regardless of whether a first PEG precipitation reaction is performed. In some embodiments, this step includes substantially precipitating Factor H from the composition, optionally, under conditions in which at least one impurity (e.g., a lipid or non-Factor H protein) in the composition is not co-precipitated, and then separating the resulting precipitate containing Factor H from the supernatant.

In some embodiments, the second PEG precipitation step is accomplished by incubating the Factor H composition, after addition of PEG (e.g., PEG 4 k) to a final concentration of at least about 10%, at a pH of from about 7.0 to about 9.0. In some embodiments, precipitation of a particular impurity of interest may be minimized by performing the reaction at a pH substantially far from the isoelectric point of the impurity.

In some embodiments, the concentration of PEG used in the reaction is adjusted to maximize the precipitation of Factor H and/or minimize the precipitation of one or more impurities. In some embodiments, the first PEG precipitation step is performed using a final PEG concentration of from about 10% to about 20%. In some embodiments, the final PEG concentration is from about 10% to about 17%, from about 10% to about 15%, or at about 10%, 11%, 12%, 13%, 14%, 15%, 16%, 17%, 18%, 19%, 20%, or more PEG. In a specific embodiment, the final PEG concentration is at or about 12%. In some embodiments, the PEG is PEG lk, PEG 3350, PEG 4 k, PEG 10 k, PEG 12 k, PEG 15 k, PEG 20 k, PEG 30 k, or PEG 40 k. In a particular embodiment, the PEG is PEG 4 k.

In some embodiments, the pH of the reaction is adjusted to maximize the precipitation of Factor H and/or minimize the precipitation of one or more impurities. In certain embodiments, the pH of the solution is adjusted to about 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, or 9.0. In one embodiment, the pH of the solution is adjusted to from about 7.2 to about 8.8. In another embodiment, the pH of the solution is adjusted to from about 7.3 to about 8.7. In another embodiment, the pH of the solution is adjusted to from about 7.4 to about 8.6. In another embodiment, the pH of the solution is adjusted to from about 7.5 to about 8.5. In another embodiment, the pH of the solution is adjusted to from about 7.6 to about 8.4. In another embodiment, the pH of the solution is adjusted from about 7.7 to about 8.3. In another embodiment, the pH of the solution is adjusted to from about 7.8 to about 8.2. In another embodiment, the pH of the solution is adjusted to from about 7.9 to about 8.1. In another embodiment, the pH of the solution is adjusted to at or about 8.0.

In one embodiment, the first PEG precipitation is performed using a final concentration of about 10% to about 20% PEG at a pH of from about 7.0 to about 9.0. In one embodiment, the first PEG precipitation is performed using a final concentration of about 10% to about 20% PEG at a pH of from about 7.5 to about 8.5. In one embodiment, the first PEG precipitation is performed using a final concentration of about 10% to about 20% PEG at a pH of 8.0±0.2. In one embodiment, the first PEG precipitation is performed using a final concentration of about 10% to about 17% PEG at a pH of from about 7.0 to about 9.0. In one embodiment, the first PEG precipitation is performed using a final concentration of about 10% to about 17% PEG at a pH of from about 7.5 to about 8.5. In one embodiment, the first PEG precipitation is performed using a final concentration of about 10% to about 17% PEG at a pH of 8.0±0.2. In one embodiment, the first PEG precipitation is performed using a final concentration of about 10% to about 15% PEG at a pH of from about 7.0 to about 9.0. In one embodiment, the first PEG precipitation is performed using a final concentration of about 10% to about 15% PEG at a pH of from about 7.5 to about 8.5. In one embodiment, the first PEG precipitation is performed using a final concentration of about 10% to about 15% PEG at a pH of 8.0±0.2. In one embodiment, the first PEG precipitation is performed using a final concentration of 12±1% PEG at a pH of from about 7.0 to about 9.0. In one embodiment, the first PEG precipitation is performed using a final concentration of 12±1% PEG at a pH of from about 7.5 to about 8.5. In one embodiment, the first PEG precipitation is performed using a final concentration of 12±1% PEG at a pH of 8.0±0.2. In one embodiment, the first PEG precipitation is performed using a final PEG concentration and pH combination selected from variations 851 to 1700 in Table 2.

TABLE 2
Useful combinations of PEG concentration and pH for the second PEG precipitation step.

| | Final PEG Percentage | | | | | | | | | | | | | | | | |
|---|---|---|---|---|---|---|---|---|---|---|---|---|---|---|---|---|---|
| pH | 12 ± 0.5% | 12 ± 1.0% | 12 ± 2% | 13 ± 2% | 14 ± 2% | 15 ± 2% | 16 ± 2% | 17 ± 2% | 18 ± 2% | 11 ± 1% | 12 ± 1% | 14 ± 1% | 15 ± 1% | 16 ± 1% | 17 ± 1% | 18 ± 1% | 19 ± 1% |
| 8.0 ± 1.0 | Var. | Var. | Var. | Var. | Var. | Var. | Var. | Var. | Var. | Var. | Var. | Var. | Var. | Var. | Var. | Var. | Var. |
| | 851 | 901 | 951 | 1001 | 1051 | 1101 | 1151 | 1201 | 1251 | 1301 | 1351 | 1401 | 1451 | 1501 | 1551 | 1601 | 1651 |
| 8.0 ± 0.9 | Var. | Var. | Var. | Var. | Var. | Var. | Var. | Var. | Var. | Var. | Var. | Var. | Var. | Var. | Var. | Var. | Var. |
| | 852 | 902 | 952 | 1002 | 1052 | 1102 | 1152 | 1202 | 1252 | 1302 | 1352 | 1402 | 1452 | 1502 | 1552 | 1602 | 1652 |
| 8.0 ± 0.8 | Var. | Var. | Var. | Var. | Var. | Var. | Var. | Var. | Var. | Var. | Var. | Var. | Var. | Var. | Var. | Var. | Var. |
| | 853 | 903 | 953 | 1003 | 1053 | 1103 | 1153 | 1203 | 1253 | 1303 | 1353 | 1403 | 1453 | 1503 | 1553 | 1603 | 1653 |
| 8.0 ± 0.7 | Var. | Var. | Var. | Var. | Var. | Var. | Var. | Var. | Var. | Var. | Var. | Var. | Var. | Var. | Var. | Var. | Var. |
| | 854 | 904 | 954 | 1004 | 1054 | 1104 | 1154 | 1204 | 1254 | 1304 | 1354 | 1404 | 1454 | 1504 | 1554 | 1604 | 1654 |
| 8.0 ± 0.6 | Var. | Var. | Var. | Var. | Var. | Var. | Var. | Var. | Var. | Var. | Var. | Var. | Var. | Var. | Var. | Var. | Var. |
| | 855 | 905 | 955 | 1005 | 1055 | 1105 | 1155 | 1205 | 1255 | 1305 | 1355 | 1405 | 1455 | 1505 | 1555 | 1605 | 1655 |
| 8.0 ± 0.5 | Var. | Var. | Var. | Var. | Var. | Var. | Var. | Var. | Var. | Var. | Var. | Var. | Var. | Var. | Var. | Var. | Var. |
| | 856 | 906 | 956 | 1006 | 1056 | 1106 | 1156 | 1206 | 1256 | 1306 | 1356 | 1406 | 1456 | 1506 | 1556 | 1606 | 1656 |
| 8.0 ± 0.4 | Var. | Var. | Var. | Var. | Var. | Var. | Var. | Var. | Var. | Var. | Var. | Var. | Var. | Var. | Var. | Var. | Var. |
| | 857 | 907 | 957 | 1007 | 1057 | 1107 | 1157 | 1207 | 1257 | 1307 | 1357 | 1407 | 1457 | 1507 | 1557 | 1607 | 1657 |
| 8.0 ± 0.3 | Var. | Var. | Var. | Var. | Var. | Var. | Var. | Var. | Var. | Var. | Var. | Var. | Var. | Var. | Var. | Var. | Var. |
| | 858 | 908 | 958 | 1008 | 1058 | 1108 | 1158 | 1208 | 1258 | 1308 | 1358 | 1408 | 1458 | 1508 | 1558 | 1608 | 1658 |
| 8.0 ± 0.2 | Var. | Var. | Var. | Var. | Var. | Var. | Var. | Var. | Var. | Var. | Var. | Var. | Var. | Var. | Var. | Var. | Var. |
| | 859 | 909 | 959 | 1009 | 1059 | 1109 | 1159 | 1209 | 1259 | 1309 | 1359 | 1409 | 1459 | 1509 | 1559 | 1609 | 1659 |
| 8.0 ± 0.1 | Var. | Var. | Var. | Var. | Var. | Var. | Var. | Var. | Var. | Var. | Var. | Var. | Var. | Var. | Var. | Var. | Var. |
| | 860 | 910 | 960 | 1010 | 1060 | 1110 | 1160 | 1210 | 1260 | 1310 | 1360 | 1410 | 1460 | 1510 | 1560 | 1610 | 1660 |
| 8.0 | Var. | Var. | Var. | Var. | Var. | Var. | Var. | Var. | Var. | Var. | Var. | Var. | Var. | Var. | Var. | Var. | Var. |
| | 861 | 911 | 961 | 1011 | 1061 | 1111 | 1161 | 1211 | 1261 | 1311 | 1361 | 1411 | 1461 | 1511 | 1561 | 1611 | 1661 |
| 7.0 ± 0.2 | Var. | Var. | Var. | Var. | Var. | Var. | Var. | Var. | Var. | Var. | Var. | Var. | Var. | Var. | Var. | Var. | Var. |
| | 862 | 912 | 962 | 1012 | 1062 | 1112 | 1162 | 1212 | 1262 | 1312 | 1362 | 1412 | 1462 | 1512 | 1562 | 1612 | 1662 |
| 7.1 ± 0.2 | Var. | Var. | Var. | Var. | Var. | Var. | Var. | Var. | Var. | Var. | Var. | Var. | Var. | Var. | Var. | Var. | Var. |
| | 863 | 913 | 963 | 1013 | 1063 | 1113 | 1163 | 1213 | 1263 | 1313 | 1363 | 1413 | 1463 | 1513 | 1563 | 1613 | 1663 |
| 7.2 ± 0.2 | Var. | Var. | Var. | Var. | Var. | Var. | Var. | Var. | Var. | Var. | Var. | Var. | Var. | Var. | Var. | Var. | Var. |
| | 864 | 914 | 964 | 1014 | 1064 | 1114 | 1164 | 1214 | 1264 | 1314 | 1364 | 1414 | 1464 | 1514 | 1564 | 1614 | 1664 |
| 7.3 ± 0.2 | Var. | Var. | Var. | Var. | Var. | Var. | Var. | Var. | Var. | Var. | Var. | Var. | Var. | Var. | Var. | Var. | Var. |
| | 865 | 915 | 965 | 1015 | 1065 | 1115 | 1165 | 1215 | 1265 | 1315 | 1365 | 1415 | 1465 | 1515 | 1565 | 1615 | 1665 |
| 7.4 ± 0.2 | Var. | Var. | Var. | Var. | Var. | Var. | Var. | Var. | Var. | Var. | Var. | Var. | Var. | Var. | Var. | Var. | Var. |
| | 866 | 916 | 966 | 1016 | 1066 | 1116 | 1166 | 1216 | 1266 | 1316 | 1366 | 1416 | 1466 | 1516 | 1566 | 1616 | 1666 |
| 7.5 ± 0.2 | Var. | Var. | Var. | Var. | Var. | Var. | Var. | Var. | Var. | Var. | Var. | Var. | Var. | Var. | Var. | Var. | Var. |
| | 867 | 917 | 967 | 1017 | 1067 | 1117 | 1167 | 1217 | 1267 | 1317 | 1367 | 1417 | 1467 | 1517 | 1567 | 1617 | 1667 |
| 7.6 ± 0.2 | Var. | Var. | Var. | Var. | Var. | Var. | Var. | Var. | Var. | Var. | Var. | Var. | Var. | Var. | Var. | Var. | Var. |
| | 868 | 918 | 968 | 1018 | 1068 | 1118 | 1168 | 1218 | 1268 | 1318 | 1368 | 1418 | 1468 | 1518 | 1568 | 1618 | 1668 |
| 7.7 ± 0.2 | Var. | Var. | Var. | Var. | Var. | Var. | Var. | Var. | Var. | Var. | Var. | Var. | Var. | Var. | Var. | Var. | Var. |
| | 869 | 919 | 969 | 1019 | 1069 | 1119 | 1169 | 1219 | 1269 | 1319 | 1369 | 1419 | 1469 | 1519 | 1569 | 1619 | 1669 |
| 7.8 ± 0.2 | Var. | Var. | Var. | Var. | Var. | Var. | Var. | Var. | Var. | Var. | Var. | Var. | Var. | Var. | Var. | Var. | Var. |
| | 870 | 920 | 970 | 1020 | 1070 | 1120 | 1170 | 1220 | 1270 | 1320 | 1370 | 1420 | 1470 | 1520 | 1570 | 1620 | 1670 |
| 7.9 ± 0.2 | Var. | Var. | Var. | Var. | Var. | Var. | Var. | Var. | Var. | Var. | Var. | Var. | Var. | Var. | Var. | Var. | Var. |
| | 871 | 921 | 971 | 1021 | 1071 | 1121 | 1171 | 1221 | 1271 | 1321 | 1371 | 1421 | 1471 | 1521 | 1571 | 1621 | 1671 |
| 8.1 ± 0.2 | Var. | Var. | Var. | Var. | Var. | Var. | Var. | Var. | Var. | Var. | Var. | Var. | Var. | Var. | Var. | Var. | Var. |
| | 872 | 922 | 972 | 1022 | 1072 | 1122 | 1172 | 1222 | 1272 | 1322 | 1372 | 1422 | 1472 | 1522 | 1572 | 1622 | 1672 |
| 8.2 ± 0.2 | Var. | Var. | Var. | Var. | Var. | Var. | Var. | Var. | Var. | Var. | Var. | Var. | Var. | Var. | Var. | Var. | Var. |
| | 873 | 923 | 973 | 1023 | 1073 | 1123 | 1173 | 1223 | 1273 | 1323 | 1373 | 1423 | 1473 | 1523 | 1573 | 1623 | 1673 |
| 8.3 ± 0.2 | Var. | Var. | Var. | Var. | Var. | Var. | Var. | Var. | Var. | Var. | Var. | Var. | Var. | Var. | Var. | Var. | Var. |
| | 874 | 924 | 974 | 1024 | 1074 | 1124 | 1174 | 1224 | 1274 | 1324 | 1374 | 1424 | 1474 | 1524 | 1574 | 1624 | 1674 |
| 8.4 ± 0.2 | Var. | Var. | Var. | Var. | Var. | Var. | Var. | Var. | Var. | Var. | Var. | Var. | Var. | Var. | Var. | Var. | Var. |
| | 875 | 925 | 975 | 1025 | 1075 | 1125 | 1175 | 1225 | 1275 | 1325 | 1375 | 1425 | 1475 | 1525 | 1575 | 1625 | 1675 |
| 8.5 ± 0.2 | Var. | Var. | Var. | Var. | Var. | Var. | Var. | Var. | Var. | Var. | Var. | Var. | Var. | Var. | Var. | Var. | Var. |
| | 876 | 926 | 976 | 1026 | 1076 | 1126 | 1176 | 1226 | 1276 | 1326 | 1376 | 1426 | 1476 | 1526 | 1576 | 1626 | 1676 |
| 8.6 ± 0.2 | Var. | Var. | Var. | Var. | Var. | Var. | Var. | Var. | Var. | Var. | Var. | Var. | Var. | Var. | Var. | Var. | Var. |
| | 877 | 927 | 977 | 1027 | 1077 | 1127 | 1177 | 1227 | 1277 | 1327 | 1377 | 1427 | 1477 | 1527 | 1577 | 1627 | 1677 |
| 8.7 ± 0.2 | Var. | Var. | Var. | Var. | Var. | Var. | Var. | Var. | Var. | Var. | Var. | Var. | Var. | Var. | Var. | Var. | Var. |
| | 878 | 928 | 978 | 1028 | 1078 | 1128 | 1178 | 1228 | 1278 | 1328 | 1378 | 1428 | 1478 | 1528 | 1578 | 1628 | 1678 |
| 8.8 ± 0.2 | Var. | Var. | Var. | Var. | Var. | Var. | Var. | Var. | Var. | Var. | Var. | Var. | Var. | Var. | Var. | Var. | Var. |
| | 879 | 929 | 979 | 1029 | 1079 | 1129 | 1179 | 1229 | 1279 | 1329 | 1379 | 1429 | 1479 | 1529 | 1579 | 1629 | 1679 |
| 8.9 ± 0.2 | Var. | Var. | Var. | Var. | Var. | Var. | Var. | Var. | Var. | Var. | Var. | Var. | Var. | Var. | Var. | Var. | Var. |
| | 880 | 930 | 980 | 1030 | 1080 | 1130 | 1180 | 1230 | 1280 | 1330 | 1380 | 1430 | 1480 | 1530 | 1580 | 1630 | 1680 |
| 9.0 ± 0.2 | Var. | Var. | Var. | Var. | Var. | Var. | Var. | Var. | Var. | Var. | Var. | Var. | Var. | Var. | Var. | Var. | Var. |
| | 881 | 931 | 981 | 1031 | 1081 | 1131 | 1181 | 1231 | 1281 | 1331 | 1381 | 1431 | 1481 | 1531 | 1581 | 1631 | 1681 |
| 7.1 ± 0.1 | Var. | Var. | Var. | Var. | Var. | Var. | Var. | Var. | Var. | Var. | Var. | Var. | Var. | Var. | Var. | Var. | Var. |
| | 882 | 932 | 982 | 1032 | 1082 | 1132 | 1182 | 1232 | 1282 | 1332 | 1382 | 1432 | 1482 | 1532 | 1582 | 1632 | 1682 |
| 7.2 ± 0.1 | Var. | Var. | Var. | Var. | Var. | Var. | Var. | Var. | Var. | Var. | Var. | Var. | Var. | Var. | Var. | Var. | Var. |
| | 883 | 933 | 983 | 1033 | 1083 | 1133 | 1183 | 1233 | 1283 | 1333 | 1383 | 1433 | 1483 | 1533 | 1583 | 1633 | 1683 |
| 7.3 ± 0.1 | Var. | Var. | Var. | Var. | Var. | Var. | Var. | Var. | Var. | Var. | Var. | Var. | Var. | Var. | Var. | Var. | Var. |
| | 884 | 934 | 984 | 1034 | 1084 | 1134 | 1184 | 1234 | 1284 | 1334 | 1384 | 1434 | 1484 | 1534 | 1584 | 1634 | 1684 |
| 7.4 ± 0.1 | Var. | Var. | Var. | Var. | Var. | Var. | Var. | Var. | Var. | Var. | Var. | Var. | Var. | Var. | Var. | Var. | Var. |
| | 885 | 935 | 985 | 1035 | 1085 | 1135 | 1185 | 1235 | 1285 | 1335 | 1385 | 1435 | 1485 | 1535 | 1585 | 1635 | 1685 |
| 7.5 ± 0.1 | Var. | Var. | Var. | Var. | Var. | Var. | Var. | Var. | Var. | Var. | Var. | Var. | Var. | Var. | Var. | Var. | Var. |
| | 886 | 936 | 986 | 1036 | 1086 | 1136 | 1186 | 1236 | 1286 | 1336 | 1386 | 1436 | 1486 | 1536 | 1586 | 1636 | 1686 |
| 7.6 ± 0.1 | Var. | Var. | Var. | Var. | Var. | Var. | Var. | Var. | Var. | Var. | Var. | Var. | Var. | Var. | Var. | Var. | Var. |
| | 887 | 937 | 987 | 1037 | 1087 | 1137 | 1187 | 1237 | 1287 | 1337 | 1387 | 1437 | 1487 | 1537 | 1587 | 1637 | 1687 |
| 7.7 ± 0.1 | Var. | Var. | Var. | Var. | Var. | Var. | Var. | Var. | Var. | Var. | Var. | Var. | Var. | Var. | Var. | Var. | Var. |
| | 888 | 938 | 988 | 1038 | 1088 | 1138 | 1188 | 1238 | 1288 | 1338 | 1388 | 1438 | 1488 | 1538 | 1588 | 1638 | 1688 |
| 7.8 ± 0.1 | Var. | Var. | Var. | Var. | Var. | Var. | Var. | Var. | Var. | Var. | Var. | Var. | Var. | Var. | Var. | Var. | Var. |
| | 889 | 939 | 989 | 1039 | 1089 | 1139 | 1189 | 1239 | 1289 | 1339 | 1389 | 1439 | 1489 | 1539 | 1589 | 1639 | 1689 |
| 7.9 ± 0.1 | Var. | Var. | Var. | Var. | Var. | Var. | Var. | Var. | Var. | Var. | Var. | Var. | Var. | Var. | Var. | Var. | Var. |
| | 890 | 940 | 990 | 1040 | 1090 | 1140 | 1190 | 1240 | 1290 | 1340 | 1390 | 1440 | 1490 | 1540 | 1590 | 1640 | 1690 |
| 8.1 ± 0.1 | Var. | Var. | Var. | Var. | Var. | Var. | Var. | Var. | Var. | Var. | Var. | Var. | Var. | Var. | Var. | Var. | Var. |
| | 891 | 941 | 991 | 1041 | 1091 | 1141 | 1191 | 1241 | 1291 | 1341 | 1391 | 1441 | 1491 | 1541 | 1591 | 1641 | 1691 |
| 8.2 ± 0.1 | Var. | Var. | Var. | Var. | Var. | Var. | Var. | Var. | Var. | Var. | Var. | Var. | Var. | Var. | Var. | Var. | Var. |
| | 892 | 942 | 992 | 1042 | 1092 | 1142 | 1192 | 1242 | 1292 | 1342 | 1392 | 1442 | 1492 | 1542 | 1592 | 1642 | 1692 |
| 8.3 ± 0.1 | Var. | Var. | Var. | Var. | Var. | Var. | Var. | Var. | Var. | Var. | Var. | Var. | Var. | Var. | Var. | Var. | Var. |
| | 893 | 943 | 993 | 1043 | 1093 | 1143 | 1193 | 1243 | 1293 | 1343 | 1393 | 1443 | 1493 | 1543 | 1593 | 1643 | 1693 |
| 8.4 ± 0.1 | Var. | Var. | Var. | Var. | Var. | Var. | Var. | Var. | Var. | Var. | Var. | Var. | Var. | Var. | Var. | Var. | Var. |
| | 894 | 944 | 994 | 1044 | 1094 | 1144 | 1194 | 1244 | 1294 | 1344 | 1394 | 1444 | 1494 | 1544 | 1594 | 1644 | 1694 |
| 8.5 ± 0.1 | Var. | Var. | Var. | Var. | Var. | Var. | Var. | Var. | Var. | Var. | Var. | Var. | Var. | Var. | Var. | Var. | Var. |
| | 895 | 945 | 995 | 1045 | 1095 | 1145 | 1195 | 1245 | 1295 | 1345 | 1395 | 1445 | 1495 | 1545 | 1595 | 1645 | 1695 |
| 8.6 ± 0.1 | Var. | Var. | Var. | Var. | Var. | Var. | Var. | Var. | Var. | Var. | Var. | Var. | Var. | Var. | Var. | Var. | Var. |
| | 896 | 946 | 996 | 1046 | 1096 | 1146 | 1196 | 1246 | 1296 | 1346 | 1396 | 1446 | 1496 | 1546 | 1596 | 1646 | 1696 |
| 8.7 ± 0.1 | Var. | Var. | Var. | Var. | Var. | Var. | Var. | Var. | Var. | Var. | Var. | Var. | Var. | Var. | Var. | Var. | Var. |
| | 897 | 947 | 997 | 1047 | 1097 | 1147 | 1197 | 1247 | 1297 | 1347 | 1397 | 1447 | 1497 | 1547 | 1597 | 1647 | 1697 |
| 8.8 ± 0.1 | Var. | Var. | Var. | Var. | Var. | Var. | Var. | Var. | Var. | Var. | Var. | Var. | Var. | Var. | Var. | Var. | Var. |
| | 898 | 948 | 998 | 1048 | 1098 | 1148 | 1198 | 1248 | 1298 | 1348 | 1398 | 1448 | 1498 | 1548 | 1598 | 1648 | 1698 |
| 8.9 ± 0.1 | Var. | Var. | Var. | Var. | Var. | Var. | Var. | Var. | Var. | Var. | Var. | Var. | Var. | Var. | Var. | Var. | Var. |
| | 899 | 949 | 999 | 1049 | 1099 | 1149 | 1199 | 1249 | 1299 | 1349 | 1399 | 1449 | 1499 | 1549 | 1599 | 1649 | 1699 |
| 9.0 ± 0.1 | Var. | Var. | Var. | Var. | Var. | Var. | Var. | Var. | Var. | Var. | Var. | Var. | Var. | Var. | Var. | Var. | Var. |
| | 900 | 950 | 1000 | 1050 | 1100 | 1150 | 1200 | 1250 | 1300 | 1350 | 1400 | 1450 | 1500 | 1550 | 1600 | 1650 | 1700 |

Var. = Variation

In one embodiment, the first PEG precipitation step is performed using PEG having an average molecular weight of from 1 kD to 20 kD and a final concentration and pH combination selected from variations 851 to 1700 in Table 2. In one embodiment, the first PEG precipitation step is performed using PEG having an average molecular weight of from 1 kD to 10 kD and a final concentration and pH combination selected from variations 851 to 1700 in Table 2. In one embodiment, the first PEG precipitation step is performed using PEG having an average molecular weight of from 1 kD to 7.5 kD and a final concentration and pH combination selected from variations 851 to 1700 in Table 2. In one embodiment, the first PEG precipitation step is performed using PEG having an average molecular weight of from 2 kD to 20 kD and a final concentration and pH combination selected from variations 851 to 1700 in Table 2. In one embodiment, the first PEG precipitation step is performed using PEG having an average molecular weight of from 2 kD to 20 kD and a final concentration and pH combination selected from variations 851 to 1700 in Table 2. In one embodiment, the first PEG precipitation step is performed using PEG having an average molecular weight of from 2 kD to 10 kD and a final concentration and pH combination selected from variations 851 to 1700 in Table 2. In one embodiment, the first PEG precipitation step is performed using PEG having an average molecular weight of from 2 kD to 7.5 kD and a final concentration and pH combination selected from variations 851 to 1700 in Table 2. In one embodiment, the first PEG precipitation step is performed using PEG having an average molecular weight of from 2 kD to 5 kD and a final concentration and pH combination selected from variations 851 to 1700 in Table 2. In one embodiment, the first PEG precipitation step is performed using PEG having an average molecular weight of from 3.35 kD to 20 kD and a final concentration and pH combination selected from variations 851 to 1700 in Table 2. In one embodiment, the first PEG precipitation step is performed using PEG having an average molecular weight of from 3.35 kD to 10 kD and a final concentration and pH combination selected from variations 851 to 1700 in Table 2. In one embodiment, the first PEG precipitation step is performed using PEG having an average molecular weight of from 3.35 kD to 7.5 kD and a final concentration and pH combination selected from variations 851 to 1700 in Table 2. In one embodiment, the first PEG precipitation step is performed using PEG having an average molecular weight of from 3.35 kD to 5 kD and a final concentration and pH combination selected from variations 851 to 1700 in Table 2. In one embodiment, the first PEG precipitation step is performed using PEG having an average molecular weight of 4±1 kD and a final concentration and pH combination selected from variations 851 to 1700 in Table 2. In one embodiment, the first PEG precipitation step is performed using PEG having an average molecular weight of 4±2 kD and a final concentration and pH combination selected from variations 851 to 1700 in Table 2.

Depending on factors including, but not limited to, the particular PEG used, a desired concentration of PEG, a desired pH for the reaction, and a desired precipitation profile, one of skill in the art will readily be able to modify the precipitation reactions described herein to achieve a desired result (e.g., balancing maximal precipitation of Factor H with minimal precipitation of targeted impurities).

F. Extraction of Factor H from Second PEG Precipitation

To extract Factor H from the second PEG precipitate, an extraction buffer is used to suspend the precipitate. In some embodiments, suspension includes addition of the extraction buffer to the second PEG precipitate followed by stirring (e.g., by hand, with stir bar, or with an impeller). In some embodiments, extracting Factor H includes mechanically breaking down the second PEG precipitate, before or after addition of the extraction buffer. Examples of techniques that can be used to break down the second PEG precipitate include, without limitation, cutting the precipitate into small pieces (e.g., with one or more blades), mashing the precipitate, blending the precipitate, grinding the precipitate, and using pressure or sonication to homogenize the precipitate. Methods for implementing these strategies are known in the art.

In some embodiments, the second PEG precipitate is suspended at a ratio of 1 part precipitate to from about 4 parts to about 40 parts extraction buffer. Other suitable ranges for the suspension ratio include, without limitation, from about 1:8 to about 1:30, from about 1:10 to about 1:20, from about 1:12 to about 1:18, from about 1:13 to about 1:17, and from about 1:14 to about 1:16. Some embodiments, suspension ratio is about 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, or higher. In a specific embodiment, the suspension ratio is about 1 part precipitate to about 15 parts suspension buffer.

In another specific embodiment, the suspension ratio is about 1 part precipitate to about 20 parts suspension buffer.

In some embodiments, the Factor H extraction buffer has a pH between about 4.0 and about 10. In some embodiments, the Factor H extraction buffer has a pH of 5.0±1, 5.5±1, 6.0±1, 6.5±1, 7.0±1, 7.5±1, 8.0±1, 8.5±1, 9.0±1, 6.0±2, 6.5±2, 7.0±2, 7.5±2, or 8.0±2. In some embodiments, the Factor H extraction buffer has a pH above or below (e.g., by at least 0.3 pH units) the isoelectric point of Factor H, determined to be 6.0 (Nagaki et al, 1978; Pio et al, 2001; and Brandstatter et al, (2012)). In some embodiments, the pH of the extraction buffer is from about 6.5 to about 10. In some embodiments, the Factor H extraction buffer has a pH of 7.0±0.5, 7.5±0.5, 8.0±0.5, 8.5±0.5, 9.0±0.5, 9.5±0.5. In some embodiments, the pH of the extraction buffer is from about 4.0 to about 5.5. In some embodiments, the Factor H extraction buffer has a pH of 4.5±0.5 or 5.0±0.5.

Generally, these pH requirements can be met using a buffering agent, including without limitation, acetate, citrate, monobasic phosphate, dibasic phosphate, mixtures thereof, and the like. Suitable buffer concentrations typically range from about 2.5 mM to about 100 mM, or from about 5 mM to about 50 mM, or about 2.5 mM, 5 mM, 10 mM, 15 mM, 20 mM, 25 mM, 50 mM, 75 mM, 100 mM, 125 mM, 150 mM, 175 mM, or 200 mM buffering agent.

In some embodiments, the extraction buffer has a conductivity equal to that of a solution of from about 50 to about 500 mM sodium chloride. In some embodiments, the extraction buffer has a conductivity equal to that of a solution of from about 100 to about 300 mM sodium chloride. In a specific embodiment, the extraction buffer has a conductivity equal to that of a solution of from about 150 to about 200 mM sodium chloride.

In some embodiments, the extraction buffer includes from 10 to 100 mM buffering agent (e.g., Tris), from 150 to 250 mM of an alkaline metal chloride salt (e.g., sodium chloride), optionally, from 1 to 10 mM of a metal chelating agent (e.g., EDTA and/or EGTA), and a pH from 7.5 to 8.5. In a specific embodiment, the extraction buffer includes 20 to 40 mM Tris, 175 to 225 mM sodium chloride, 5±1 mM EDTA, and a pH of 8.0±0.2.

Generally, the extraction is performed at a temperature of from about 0° C. and about 25° C. In certain embodiments, the extraction is performed at about 0° C., 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., or 25° C. In a particular embodiment, the extraction is performed at around room temperature (about 20° C.). In another particular embodiment, the extraction is performed at from about 2° C. to about 10° C. In some embodiments, the extraction process is performed under continuous agitation (e.g., stirring, cutting, mashing, blending, grinding, or otherwise homogenizing) until all soluble components of the second PEG precipitate are brought into solution. In certain embodiments, the extraction will proceed for at or about between 30 and 300 minutes, or for at or between 120 and 240 min, or for at or about between 150 and 210 minutes. In certain embodiments, the extraction process will proceed for about 30, 45, 60, 90, 120, 150, 180, 210, 240, 270, 300, or more minutes, optionally, with continuous agitation.

G. Further Enrichment of Factor H

In some embodiments, the method for preparing an enriched Factor H composition includes at least one, preferably two or more, chromatographic steps to further enrich the purity of the composition. Generally, any suitable chromatographic method may be employed to further enrich the Factor H composition extracted from the Fraction I precipitate.

In some embodiments, the chromatographic step includes one or more of anion exchange chromatography (AEC), cation exchange chromatography (CEC), heparin affinity chromatography, mixed-mode chromatography, hydrophobic exchange chromatography (HIC), hydroxyapatite chromatography (HAP), immuno-affinity chromatography, size exclusion chromatography (SEC), or other suitable chromatographic step. Chromatographic steps may be performed in either batch or column mode.

In one embodiment, the method includes at least anion exchange and heparin affinity chromatography. For example, in one embodiment, the method includes steps of: binding Factor H to an anion exchange resin; eluting Factor H from the anion exchange resin with an elution buffer, thereby forming an anion exchange eluate containing Factor H; binding Factor H to a heparin affinity resin; and eluting Factor H from the heparin affinity resin with an elution buffer, thereby forming a heparin affinity eluate.

In some embodiments, the anion exchange step is performed prior to the heparin affinity step. In some embodiments, the heparin affinity step is performed prior to the anion exchange step. In some embodiments, the starting material for the chromatographic steps is a first PEG supernatant. In some embodiments, the starting material for the chromatographic steps is a second PEG precipitate suspension. In some embodiments, the chromatographic methods may include wash steps to remove loosely bound impurities from the chromatographic resin prior to elution of Factor H. In certain embodiments, Factor H may be eluted from a chromatography resin by gradient elution (e.g., with a salt gradient) or by step elution (e.g., with buffers having increasing conductivity/ionic strength).

Generally, the conductivity of the Factor H solution is adjusted to an appropriate conductivity/ionic strength prior to binding Factor H onto a chromatographic resin. The conductivity/ionic strength should be selected appropriately to promote the interaction between Factor H and the resin. The requirements for the conductivity/ionic strength of the solution will vary dependent upon factors such as the identity of the resin used (e.g., strong vs. weak anion exchange resin) and the starting purity of the solution. Various methods may be employed for reducing the conductivity/ionic strength of a Factor H composition, including without limitation, dilution of the composition with a solution having a low conductivity/ionic strength, precipitating Factor H from the starting composition and re-suspending in a buffer having lower conductivity/ionic strength, ultrafiltration/diafiltration, desalting and/or buffer exchange chromatography, and dialysis.

Any suitable anion exchange resin may be used in the methods provided herein. Non-limiting examples of anion exchange resins suitable for use include diethylaminoethyl (DEAE), quaternary aminoethyl (QAE), and quaternary ammonium (O) resins. In a preferred embodiment, the anion exchange resin used is DEAE Sepharose™ (diethylaminoethyl-Sepharose).

In some embodiments, Factor H is bound to a DEAE Sepharose™ resin in the presence of a low conductivity/ionic strength loading buffer. Typically, the column will be equilibrated with the same loading buffer or a compatible buffer with a conductivity/ionic strength similar to the loading buffer. In certain embodiments, the loading and/or equilibration buffer will have a conductivity of less than at or about 12 mS/cm. In some embodiments, the loading and/or equilibration buffer will have a conductivity of less than at or about 10 mS/cm. In some embodiments, the loading and/or equilibration buffer will have a conductivity of at or about 9 mS/cm. In some embodiments, the loading and/or equilibration buffer will have a salt concentration of no more than about 100 mM NaCl, or conductivity/ionic strength corresponding to no more that of a 100 mM NaCl solution. In some embodiments, the loading and/or equilibration buffer will have a salt concentration, or corresponding conductivity/ionic strength, of no more than about 75 mM NaCl. In some embodiments, the salt concentration, or corresponding conductivity/ionic strength, is from about 30 to about 70 mM NaCl. In a specific embodiment, the salt concentration, or corresponding conductivity/ionic strength, is from about 50 mM to about 65 mM NaCl.

Optionally, after binding Factor H, the anion exchange resin may be washed with one or more buffers having a conductivity/ionic strength intermediate of the loading buffer and the elution buffer. In certain embodiments, a wash buffer may have a conductivity at or about between 9 mS/cm and 12.5 mS/cm. In some embodiments, the wash buffer may have a conductivity at or about between 5 mS/cm and 10 mS/cm. In some embodiments, the wash buffer will have a salt concentration, or conductivity/ionic strength corresponding to, at or about between 30 and 100 mM NaCl. In some embodiments, the wash buffer will have a salt concentration, or conductivity/ionic strength corresponding to, at or about between 40 and about 70 mM NaCl. In some embodiments, the wash buffer will have a salt concentration, or conductivity/ionic strength corresponding to, at or about 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM NaCl.

In certain embodiments, Factor H is eluted from the anion exchange resin (e.g., DEAE Sepharose™) with an elution buffer having suitable conductivity/ionic strength to disrupt the interaction between the resin and Factor H. In some embodiments, the elution buffer will not have a suitable conductivity/ionic strength to disrupt the interaction between the resin and a contaminant that binds the resin with higher affinity than does Factor H. In certain embodiments, the elution buffer will have a conductivity of at least about 12 mS/cm. In some embodiments, the elution buffer will have a conductivity of at or about 13 mS/cm. In some embodiments, the elution buffer will have a conductivity of at or about 14 mS/cm. In some embodiments, the elution buffer will have a salt concentration, or conductivity corresponding to, at least about 100 mM NaCl, preferably at least about 120 mM. In some embodiments, the elution buffer will have a salt concentration, or conductivity/ionic strength corresponding to, at least about 90 mM NaCl or at least about 95, 100, 105, 110, 115, 120, 125, 130, 140, 150 mM NaCl, or more.

Any suitable heparin affinity resin may be used in the methods provided herein, for example, resins conjugated to a heparin ligand, derivative or mimetic of a heparin ligand, or heparin-like ligand (e.g., a sulfated glycosaminoglycan). In a preferred embodiment, the heparin affinity resin used is Heparin Sepharose™

In some embodiments, Factor H (e.g., from the first PEG supernatant, the second PEG precipitate suspension, or the anion exchange eluate) is further enriched by heparin affinity chromatography, e.g., using a Heparin Sepharose™ resin. In one embodiment, the conductivity/ionic strength of the Factor H eluate is reduced by a suitable method, e.g., dilution, buffer exchange, dialysis, etc., and Factor H is bound to a heparin affinity resin. In certain embodiments, the conductivity of the anion exchange eluate is reduced to less than at or about 12 mS/cm. In some embodiments, the conductivity is reduced to less than at or about 10 mS/cm. In some embodiments, the conductivity is reduced to less than at or about 8 mS/cm. In some embodiments, the conductivity may be reduced to less than at or about 4 mS/cm, or less than at or about 5, 6, 7, 8, 9, 10, 11, or 12 mS/cm. In some embodiments, the salt concentration of the anion exchange eluate, or conductivity/ionic strength corresponding to, is reduced less than at or about 100 mM NaCl. In some embodiments, the salt concentration, or conductivity/ionic strength corresponding to, is reduced less than at or about 75 mM NaCl. In some embodiments, the salt concentration, or conductivity/ionic strength corresponding to, is reduced to less than at or about 50 mM NaCl. In some embodiments, the salt concentration, or conductivity/ionic strength corresponding to, is reduced less than at or about 20 mM NaCl, or less than at or about 25, 30, 40, 50, 60, 70, 80, 90, or 100 mM NaCl.

Optionally, after binding Factor H, the heparin affinity resin may be washed with one or more buffers having a conductivity/ionic strength intermediate of the loading buffer and the elution buffer. In certain embodiments, a wash buffer may have a conductivity at or about between 3 mS/cm and 12.5 mS/cm. In some embodiments, the wash buffer may have a conductivity at or about between 5 mS/cm and 10 mS/cm. In some embodiments, the wash buffer will have a salt concentration, or conductivity/ionic strength corresponding to, at or about between 30 and 100 mM NaCl. In some embodiments, the wash buffer will have a salt concentration, or conductivity/ionic strength corresponding to, at or about between 30 and 80 mM NaCl. In some embodiments, the wash buffer will have a salt concentration, or conductivity/ionic strength corresponding to, at or about 30, 35, 40, 45, 50, 55, 60, 65, 70, 75, 80, 85, 90, 95, or 100 mM NaCl.

In some embodiments, Factor H is eluted from the heparin affinity resin (e.g., DEAE Sepharose™) with an elution buffer having suitable conductivity/ionic strength to disrupt the interaction between the resin and Factor H. In some embodiments, the elution buffer will not have a suitable conductivity/ionic strength to disrupt the interaction between the resin and a contaminant that binds the resin with higher affinity than does Factor H. In certain embodiments, the elution buffer will have a conductivity of at least about 12 mS/cm. In certain embodiments, the elution buffer will have a salt concentration, or conductivity/ionic strength corresponding to, at least about 100 mM NaCl. In another embodiment, the elution buffer will have a salt concentration, or conductivity/ionic strength corresponding to, at least about 150 mM NaCl. In yet another embodiment, the elution buffer will have a salt concentration, or conductivity/ionic strength corresponding to, at least about 200 mM NaCl. In certain embodiments, the elution buffer will have a salt concentration, or conductivity/ionic strength corresponding to, at least about 90 mM NaCl or at least about 95 mM, 100 mM, 105 mM, 110 mM, 115 mM, 120 mM, 125 mM, 130 mM, 140 mM, 150 mM, 160 mM, 170 mM, 180 mM, 190 mM, 200 mM, 210 mM, 220 mM NaCl, or more.

In some embodiments, the method includes a cation exchange chromatographic step. In some embodiments, the cation exchange step is performed under flow through conditions, in which Factor H does not bind to the cation exchange resin. In this fashion, a Factor H composition is applied to a cation exchange chromatography column and the flow through, containing Factor H is collected. Any suitable cation exchange resin may be used in the methods provided herein, including without limitation, carboxymethyl (CM), sulfopropyl (SP), methyl sulfonate (S) resins.

Any suitable hydroxyapatite or other calcium-based resin may be used in the methods provided herein. Non-limiting examples of suitable resins include hydroxyapatite resins, fluorapatite resins, fluorhydroxyapatite resins, and the like.

Any suitable hydrophobic interaction chromatography resin may be used in the methods provided herein. Non-limiting examples of suitable resins include phenyl-resins, methyl-resins, butyl-resins, octyl-resins, and the like.

In certain embodiments, Factor H may be further enriched by immuno-affinity chromatography, for example with resins conjugated to an antibody, aptamer, or other binding molecule highly specific for Factor H.

In certain embodiments, individual or all chromatographic steps will rely on a common buffer system, in which only the salt concentration varies between the equilibration, wash, and elution buffers. Any suitable buffer may be used, e.g., a Tris buffer, a phosphate buffer, a citrate buffer, etc. The pH of the loading buffer will range at or about between 6.0 and 9.0. In some embodiments, the pH of the buffer system is at or about between 7.0 and 9.0. In some embodiments, the pH of the buffer system is at or about between 7.5 and 8.5. In some embodiments, the pH of the buffer system will be at or about 8.0.

H. Virus Removal and Inactivation

In some embodiments, the methods provided herein for the preparation of an enriched Factor H composition further include at least one, preferably at least two, more preferably three different viral inactivation or removal steps. Non-limiting examples of viral inactivation or removal steps that may be employed with the methods provided herein include, solvent detergent treatment (Horowitz et al., Blood Coagul Fibrinolysis 1994 (5 Suppl 3):S21-S28 and Kreil et al., Transfusion 2003 (43):1023-1028, the disclosures of which are hereby expressly incorporated herein by reference in their entireties for all purposes), nanofiltration (Hamamoto et al., Vox Sang 1989 (56)230-236 and Yuasa et al., J Gen Virol. 1991 (72 (pt 8)):2021-2024, the disclosures of which are hereby expressly incorporated herein by reference in their entireties for all purposes), and low pH incubation at high temperatures (Kempf et al., Transfusion 1991 (31)423-427 and Louie et al., Biologicals 1994 (22):13-19, the disclosure of which is hereby expressly incorporated herein by reference in its entirety for all purposes). In some embodiments, the methods provided herein include S/D treatment and nanofiltration steps.

Viral inactivation or removal steps may be performed on a final enriched Factor H composition and/or on any intermediate Factor H compositions generated during the manufacturing process. For example, in one embodiment, a viral inactivation or removal step may be performed on a Fraction I precipitate extract, a first PEG supernatant, a second PEG precipitate extract, an anion exchange eluate, a heparin affinity eluate, an ultrafiltration or diafiltration product, etc.

1. Solvent and Detergent (S/D) Treatment In order to inactivate various viral contaminants which may be present in plasma-derived products, one or more Factor H intermediate composition may be subjected to a solvent detergent (S/D) treatment. Methods for the detergent treatment of plasma-derived fractions are well known in the art (for review see, Pelletier J P et al., Best Pract Res Clin Haematol. 2006; 19(1):205-42, the disclosure of which is expressly incorporated by reference herein in its entirety for all purposes). Generally, any standard S/D treatment may be used in conjunction with the methods provided herein. For example, an exemplary protocol for an S/D treatment is provided below.

In some embodiments, Triton X-100, Tween-20, and tri(n-butyl)phosphate (TNBP) are added to a Factor H intermediate composition at final concentrations of at or about 1.0%, 0.3%, and 0.3%, respectively. The mixture is then stirred at a temperature of between about 18° C. and about 25° C. for at least about an hour.

In one embodiment, the S/D reagents (e.g., Triton X-100, Tween-20, and TNBP) are added by spraying rather than by fluent addition. In other embodiments, the detergent reagents may be added as solids to the Factor H intermediate solution, which is being mixed to ensure rapid distribution of the S/D components. In certain embodiments, it is preferable to add solid reagents by sprinkling the solids over a delocalized surface area of the filtrate such that local overconcentration does not occur, such as in fluent addition. In another embodiment, the Factor H containing solution is pumped into a tank where the SD-reagents are already present either in concentrated or diluted form.

2. Nanofiltration

In some embodiments, the methods provided herein include nanofiltration of an enriched Factor H composition, or an intermediate thereof, using a suitable nanofiltration device. In certain embodiments, the nanofiltration device will have a mean pore size of from about 15 nm to about 100 nm. Examples of nanofilters suitable for this use include, without limitation, DVD, DV 50, DV 20 (Pall), Viresolve NFP, Viresolve NFR (Millipore), Planova 15N, 20N, 35N, and 75N (Planova). In some embodiments, the nanofilter has a mean pore size of from about 15 nm to about 72 nm, or from about 19 nm to about 35 nm, or about 15 nm, 19 nm, 35 nm, or 72 nm. In some embodiments, the nanofilter has a mean pore size of or about 35 nm, such as an Asahi PLANOVA 35N filter, or equivalent thereof.

3. Incubation at Low pH

In some embodiments, the enriched Factor H composition, or an intermediate thereof, is incubated at low pH to reduce or inactivate the viral load of the composition. In one embodiment, this is achieved by adjusting the pH of the of the composition to low pH, for example, less than at or about 6.0, and incubating for at least about a week prior to releasing the composition. In a preferred embodiment, the pH of the bulk solution is adjusted to less than at or about 5.5 prior to incubation. In a more preferred embodiment, the pH of the solution is lowered to less than at or about 5.0 prior to incubation. In certain embodiments, the pH of the solution is lowered to less than at or about 6.0 or less than at or about 5.9, 5.8, 5.7, 5.6, 5.5, 5.4, 5.3, 5.2, 5.1, 5.0, 4.9, 4.8, 4.7, 4.6, 4.5, 4.4, 4.3, 4.2, 4.1, 4.0, or lower prior to incubation.

In some embodiments, the enriched Factor H composition, or an intermediate thereof, is incubated for at least about one week, or at least about 2, 3, 4, or more weeks, or for at least about 1, 2, 3, or more months. In some embodiments, the composition is incubated at a temperature above about 20° C., or above about 25° C., or above about 30° C. In some embodiments, the composition is incubated at a temperature of at or about 20° C., or at or about 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., or higher.

4. Lyophilization and Heat Treatment

In some embodiments, in which the enriched Factor H composition is lyophilized, the method for preparing the composition includes heat treatment of the lyophilized composition. Heat treatments for the inactivation of viruses in compositions of blood factors are well known in the art (for example, see, Piszkiewicz et al., Thromb Res. 1987 Jul. 15; 47(2):235-41; Piszkiewicz et al., Curr Stud Hematol Blood Transfus. 1989; (56):44-54; Epstein and Fricke, Arch Pathol Lab Med. 1990 March; 114(3):335-40, the disclosures of which are hereby expressly incorporated by reference in their entireties for all purposes).

I. Ultrafiltration and Diafiltration

In some embodiments, the methods provided herein include an ultrafiltration step to concentrate and/or formulate the enriched Factor H composition. In some embodiments, ultrafiltration is performed using a cassette (e.g., with an open channel screen) and an ultrafiltration membrane having a nominal molecular weight cut off (NMWCO) of no more than 150 kDa or no more than 140, 130, 120, 100, 90, 80, 70, 60, 50, 40, or 30 kDa. In some embodiments, the ultrafiltration membrane has a NMWCO of about 50 kDa. In one embodiment, the ultrafiltration membrane has a NMWCO of about 70 kDa. In one embodiment, the ultrafiltration membrane has a NMWCO of about 30 kDa. In some embodiments, the Factor H solution is concentrated to a final protein concentration of at or about between 0.5% and 25% (w/v), or at or about between 1% and 25% (w/v), or at or about between 2% and 20% (w/v), or at or about between 3% and 15% (w/v), or at or about between 5% and 10% (w/v), or at or about between 9% and 12%, or at or about between 3% and 7% (w/v), or at or about between 8% and 14% (w/v), or at or about between 4% and 6%, or to a final concentration of at or about 0.1%, 0.25%, 0.5%, 1%, 2%, 3%, 4%, 5%, or 6%, 7%, 8%, 9%, 10%, 11%, 12%, 13%, 14%, 15%, 16%, 17%, 18%, 19%, 20%, 21%, 22%, 23%, 24%, 25% or higher.

In some embodiments, prior to and/or after ultrafiltration, buffer exchange may be performed by diafiltration against a solution suitable for intravenous, intramuscular, intraocular, subcutaneous, or other appropriate route for administration of Factor H. Typically, the minimum exchange volume is at least about 3 times the original concentrate volume or at least about 4, 5, 6, 7, 8, 9, or more times the original concentrate volume.

### IV. FACTOR H COMPOSITIONS

Factor H compositions have been described for the treatment of certain complement related disorders. (See, for example, U.S. Patent Publication No. US 2009/0118163 and European Patent Application No. EP 0 222 611 A2, the disclosures of which are hereby expressly incorporated herein by reference in their entireties for all purposes.)

In one aspect, enriched Factor H compositions prepared according to any of the methods described herein are provided. In some embodiments, the enriched Factor H composition is an aqueous composition. In some embodiments, the enriched Factor H composition is formulated for pharmaceutical administration, for example by intravenous, intramuscular, intraocular, subcutaneous, or any other appropriate route for therapeutic administration of Factor H.

In some embodiments, Factor H is provided in a therapeutically effective dose between about 0.05 mg/mL and about 50 mg/mL. In some embodiments, Factor H is present at a concentration of between about 0.1 mg/mL and about 25 mg/mL. In some embodiments, Factor H is present at a concentration of between about 0.1 mg/mL and about 10 mg/mL. In some embodiments, Factor H is present at a concentration of between about 0.1 mg/mL and about 5 mg/mL. In another embodiment, Factor H is present at a concentration of between about 0.1 mg/mL and about 2 mg/mL. In another embodiment, Factor H is present at a concentration of between about 1 mg/mL and about 2 mg/mL. In yet other embodiments, Factor H may be present at about 0.01 mg/mL, or at about 0.02 mg/mL, 0.03 mg/mL, 0.04 mg/mL, 0.05 mg/mL, 0.06 mg/mL, 0.07 mg/mL, 0.08 mg/mL, 0.09 mg/mL, 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1.0 mg/mL, 1.1 mg/mL, 1.2 mg/mL, 1.3 mg/mL, 1.4 mg/mL, 1.5 mg/mL, 1.6 mg/mL, 1.7 mg/mL, 1.8 mg/mL, 1.9 mg/mL, 2.0 mg/mL, 2.5 mg/mL, 3.0 mg/mL, 3.5 mg/mL, 4.0 mg/mL, 4.5 mg/mL, 5.0 mg/mL, 5.5 mg/mL, 6.0 mg/mL, 6.5 mg/mL, 7.0 mg/mL, 7.5 mg/mL, 8.0 mg/mL, 8.5 mg/mL, 9.0 mg/mL, 9.5 mg/mL, 10.0 mg/mL, 11 mg/mL, 12 mg/mL, 13 mg/mL, 14 mg/mL, 15 mg/mL, 16 mg/mL, 17 mg/mL, 18 mg/mL, 19 mg/mL, 20 mg/mL, 22.5 mg/mL, 25 mg/mL, 27.5 mg/mL, 30 mg/mL, 35 mg/mL, 40 mg/mL, 45 mg/mL, 50 mg/mL, or a higher concentration.

In some embodiments, the concentration of a Factor H formulation may be determined by spectroscopy (e.g., total protein measured at A280) or other bulk determination (e.g., Bradford assay, silver stain, weight of a lyophilized powder, etc.). In some embodiments, the concentration of Factor H may be determined by a Factor H ELISA assay (e.g., mg/mL antigen).

In some embodiments, a pharmaceutical Factor H composition has a purity of at least 80% Factor H. In some embodiments, a pharmaceutical Factor H composition has a purity of at least 85% Factor H. In some embodiments, a pharmaceutical Factor H composition has a purity of at least 90% Factor H. In some embodiments, a pharmaceutical Factor H composition has a purity of at least 95% Factor H. In some embodiments, a pharmaceutical Factor H composition has a purity of at least 98% Factor H. In some embodiments, a pharmaceutical Factor H composition has a purity of at least 99% Factor H.

Pharmaceutical carriers and excipients as well as suitable pharmaceutical formulations are well known in the art (see, for example “Pharmaceutical Formulation Development of Peptides and Proteins”, Frokjaer et al., Taylor & Francis (2000) or “Handbook of Pharmaceutical Excipients,” 3rd edition, Kibbe et al., Pharmaceutical Press (2000)).

In some embodiments, the enriched Factor H pharmaceutical composition is formulated in lyophilized or stable soluble form. The Factor H pharmaceutical composition may be lyophilized by a variety of procedures known in the art. Lyophilized formulations are reconstituted prior to use by the addition of one or more pharmaceutically acceptable diluents such as sterile water for injection or sterile physiological saline solution.

Formulations of the enriched Factor H composition are delivered to the individual by any pharmaceutically suitable means of administration. Various delivery systems are known and can be used to administer the composition by any convenient route. In some embodiments, the compositions of the invention are administered systemically. For systemic use, in accordance with some embodiments, Factor H is formulated for parenteral (e.g. intravenous, subcutaneous, intramuscular, intraperitoneal, intracerebral, intrapulmonar, intranasal, intravitreal, or transdermal) or enteral (e.g., oral, vaginal or rectal) delivery according to conventional methods. The formulations can be administered continuously by infusion or by bolus injection. Some formulations encompass slow release systems. Preferred routes of administration will depend upon the indication being treated, managed, or prevented. For example, in one embodiment, wherein Factor H is administered for the treatment of AMD, the preferred route of administration will be intravitreal. In a second embodiment, wherein Factor H is being administered for the treatment or management of aHUS, the preferred route of administration will be intravenous. A skilled physician will readily be able to determine the preferred route of administration for the particular affliction being treated, managed, or prevented.

### V. METHODS OF TREATMENT

In one aspect, methods for treating a disease or disorder associated with a Factor H dysfunction or abnormal alternative pathway complement activity in a subject in need thereof are provided by administering a therapeutically effective dose of an enriched Factor H composition purified from Fraction I precipitate.

In some embodiments, the present disclosure provides a therapeutically effective dose of an enriched Factor H composition prepared by a method disclosed herein for use in a method for treating a disease associated with Factor H dysfunction in a subject in need thereof. In some embodiments, the disease or disorder associated with a Factor H dysfunction is selected from atypical haemolytic uremic syndrome (aHUS), age-related macular degeneration (AMD), membranoproliferative glomulonephritis type II (MPGNII), myocardial infarction, coronary heart disease/coronary artery disease (CAD/CHD), and Alzheimer's disease. In one particular embodiment, the disease is atypical haemolytic uremic syndrome (aHUS). In another particular embodiment, the disease is age-related macular degeneration (AMD). In yet another particular embodiment, the disease is membranoproliferative glomulonephritis type II (MPGNII).

In some embodiments, the present disclosure provides a therapeutically effective dose of an enriched Factor H composition prepared by a method disclosed herein for use in a method for treating a disease associated with abnormal alternative pathway complement activity in a subject in need thereof. In some embodiments, the disease or disorder associated with abnormal alternative pathway complement activity is selected from an autoimmune disease (such as rheumatoid arthritis, IgA nephropathy, asthma, systemic lupus erythematosus, multiple sclerosis, Anti-Phospholipid syndrome, ANCA-associated vasculitis, pemphigus, uveitis, myathemia gravis, Hashimoto's thyroiditis), a renal disease (such as IgA nephropathy, hemolytic uremic syndrome, membranoproliferative glomerulonephritis) asthma, Alzheimer disease, adult macular degeneration, proximal nocturnal hemoglobinuria, abdominal aortic aneurism, ischemia reperfusion injury, sepsis, and solid organ transplant.

In some embodiments, an enriched Factor H pharmaceutical composition, as provided herein, is administered alone. In some embodiments, an enriched Factor H pharmaceutical composition, as provided herein, is administered in conjunction with other therapeutic agents. In some embodiments, the additional therapeutic agents may be incorporated as part of the same pharmaceutical composition as Factor H. In some embodiments, the additional therapeutic agents may be formulated as a separate pharmaceutical composition as Factor H.

In accordance with some embodiments, the time needed to complete a course of treatment with a Factor H composition can be determined by a physician and may range from as short as one day to more than a year.

An effective amount of a Factor H preparation is administered to the subject by any suitable means to treat the disease or disorder. For example, in certain embodiments, Factor H may be administered by intravenous, intraocular, subcutaneous, and/or intramuscular means.

In a preferred embodiment, a method for treating age-related macular degeneration in a subject in need thereof is provided comprising the intraocular administration of a Factor H composition to the patient.

In certain embodiments, the Factor H compositions provided herein can be administered either systemically or locally. Systemic administration includes: oral, transdermal, subdermal, intraperitioneal, subcutaneous, transnasal, sublingual, or rectal. The most preferred systemic route of administration is oral. Local administration for ocular administration includes: topical, intravitreal, periocular, transcleral, retrobulbar, juxtascleral, sub-tenon, or via an intraocular device. Preferred methods for local delivery include transscleral delivery to the macula by posterior juxtascleral administration; via intravitreal injection; or via cannula, such as that described in U.S. Pat. No. 6,413,245, the disclosure of which is incorporated by reference herein in its entirety for all purposes. Alternatively, Factor H may be delivered via a sustained delivery device implanted intravitreally or transsclerally, or by other known means of local ocular delivery.

In certain embodiments, the term “effective amount” refers to an amount of a Factor H preparation that results in an improvement or remediation of disease or condition in the subject. An effective amount to be administered to the subject can be determined by a physician with consideration of individual differences in age, weight, the disease or condition being treated, disease severity and response to the therapy. In certain embodiments, a Factor H preparation can be administered to a subject at dose of between about 0.1 mg/kilogram and about 2000 mg/kilogram per administration. In certain embodiments, the dose may be at least at about 0.1 mg/kg, at least about 0.2 mg/kg, at least about 0.5 mg/kg, or at least about 1.0 mg/kg, 2.0 mg/kg, 3.0 mg/kg, 4.0 mg/kg, 5.0 mg/kg, 7.5 mg/kg, 10 mg/kg, 15 mg/kg, 20 mg/kg, 30 mg/kg, 40 mg/kg, 50 mg/kg, 60 mg/kg, 70 mg/kg, 80 mg/kg, 90 mg/kg, 100 mg/kg, 125 mg/kg, 150 mg/kg, 175 mg/kg, 200 mg/kg, 250 mg/kg, 300 mg/kg, 350 mg/kg, 400 mg/kg, 450 mg/kg, 500 mg/kg, 550 mg/kg, 600 mg/kg, 650 mg/kg, 700 mg/kg, 750 mg/kg, 800 mg/kg, 850 mg/kg, 900 mg/kg, 950 mg/kg, 1000 mg/kg, 1100 mg/kg, 1200 mg/kg, 1300 mg/kg, 1400 mg/kg, 1500 mg/kg, 1600 mg/kg, 1700 mg/kg, 1800 mg/kg, 1900 mg/kg, or 2000 mg/kg. The dosage and frequency of Factor H treatment will depend upon, among other factors, the disease or condition being treated and the severity of the disease or condition in the patient.

In some embodiments, Factor H is administered at an absolute dosage, rather than a dosage dependent upon the weight of the individual. For example, in some embodiments, Factor H is administered intravitreally at a dosage set based on the identity and severity of the particular indication being treated (e.g., macular degeneration or age-related macular degeneration). In some embodiments, Factor H is administered at a fixed dose of from about 0.1 mg to about 200 g. In some embodiments, Factor H is administered at a fixed dose of from about 0.1 mg to about 20 g. In some embodiments, Factor H is administered at a fixed dose of from about 0.1 mg to about 2 g. In some embodiments, Factor H is administered at a fixed dose of from about 0.1 mg to about 200 mg. In some embodiments, Factor H is administered at a fixed dose of from about 0.1 mg to about 20 mg. In some embodiments, Factor H is administered at a fixed dose of from about 0.1 mg to about 10 mg. In some embodiments, Factor H is administered at a fixed dose of from about 0.1 mg to about 5 mg. In some embodiments, Factor H is administered intravitreally at a fixed dose of about 0.1 mg to about 10 mg. In some embodiments, Factor H is administered intravitreally at a fixed dose of about 0.1 mg to about 5 mg. In some embodiments, Factor H is administered intravitreally at a fixed dose of about 1 mg to about 3 mg. In some embodiments, Factor H is administered intravitreally at a fixed dose of about 0.1 mg, 0.2 mg, 0.3 mg, 0.4 mg, 0.5 mg, 0.6 mg, 0.7 mg, 0.8 mg, 0.9 mg, 1.0 mg, 1.1 mg, 1.2 mg, 1.3 mg, 1.4 mg, 1.5 mg, 1.6 mg, 1.7 mg, 1.8 mg, 1.9 mg, 2.0 mg, 2.1 mg, 2.2 mg, 2.3 mg, 2.4 mg, 2.5 mg, 2.6, mg 2.7 mg, 2.8 mg, 2.9 mg, 3.0 mg, 2.5 mg, 4.0 mg, 4.5 mg, 5.0 mg, 5.5 mg, 6.0 mg, 6.5 mg, 7.0 mg, 7.5 mg, 8.0 mg, 8.5 mg, 9.0 mg, 9.5 mg, 10.0 mg or more.

### VI. EXAMPLES

The following examples are provided by way of illustration only and not by way of limitation. Those of skill in the art will readily recognize a variety of non-critical parameters that could be changed or modified to yield essentially the same or similar results.

### Example 1

### Purification of Factor H from Cohn Fraction II+III Silicon Dioxide Filter Cake

As reported in U.S. Pat. No. 8,304,524, Factor H can be purified from Cohn Fraction II+III silicon dioxide filter cake, a byproduct of the process used to manufacture pooled human immunoglobulin G compositions. The method used for Factor H purifications from Cohn Fraction II+III silicon dioxide filter cake in U.S. Pat. No. 8,304,524 is outlined as method 100 in FIG. 1. Briefly, method 100 includes: dissolving (102) the filter cake to extract Factor H, reducing the conductivity (102) of the dissolved solution, centrifuging and filtering (106) to clarify the dissolved solution, enriching Factor H (108) by DEAE anion exchange chromatography, reducing the conductivity (110) of the DEAE eluate, and enriching Factor H (112) by heparin affinity chromatography.

### Example 2

### Stability Characterization of Factor H Purified from Fraction II+III Silicon Dioxide Filter Cake

During the purification of Factor H from Cohn Fraction II+III silicon dioxide filter cake, as described in Example 1, it was noticed that under reducing conditions, e.g., conditions that eliminate disulfide bonds, purified Factor H resolved as two major bands during SDS-PAGE analysis, suggesting that Factor H was being proteolytically clipped (e.g., the Factor H polypeptide backbone was being broken at a discreet location). Due to the extensive network of disulfide bonds that stabilize the tertiary structure of Factor H, this proteolytic clipping was not observed under non-reducing conditions.

For example, the SDS-PAGE gel reproduced in FIG. 2 shows that under non-reducing condition (NR), the migration pattern of Factor H purified from Cohn Fraction II+III silicon dioxide filter cake (FH002 and FH004), as described in Example 1, is indistinguishable from the migration pattern of a plasma-derived Factor H standard (CompTech, Tyler, Tex.), and appears as a single band of the gel (compare Fraction II+III purified Factor H in lanes 3 and 4 to plasma-derived Factor H in lane 2). However, under reducing the conditions, the Factor H purified from Cohn Fraction II+III silicon dioxide filter cake migrates as three distinct bands (FIG. 7, lanes 8 and 9), including a doublet centered around 140 kDa (e.g., corresponding to full-length and the large fragment of the proteolytically clipped Factor H) and a single band migrating around 35 kDa (e.g., corresponding to the small fragment of the proteolytically clipped Factor H). In contrast, the plasma-derived Factor H control (FIG. 7, lane 7) migrates largely as a single band around 155 kDa (e.g., corresponding to the full-length Factor H). Without being bound by theory, the proteolytic clipping of Factor H purified from Cohn Fraction II+III silicon dioxide filter cake was attributed to amidolytic proteases known to be present in Cohn Fraction II+III silicon dioxide filter cakes.

### Example 3

### Characterization of Proteolytic Activity in Factor H Compositions Purified from Fraction II+III Silicon Dioxide Filter Cake

The source of the observed proteolytic clipping of Factor H in compositions enriched from Cohn Fraction II+III silicon dioxide filter cake was further investigated. An experiment testing the stability of Factor H upon incubation at 4° C. for an extended time demonstrated that Factor H compositions purified from Cohn Fraction II+III silicon dioxide filter cake (lots FH002 and FH004), as described in Example 1, contained proteolytic activities. Briefly, an aliquot of Factor H isolated from Cohn Fraction II+III silicon dioxide filter cake (lot FH012) was incubated for 13 days at 4° C. The structure of the incubated Factor H (FH12 13 d 4° C., lane 2) is compared in FIG. 3A to the structure of: Factor H from the same purification lot (FH012 fresh thaw, lane 3); Factor H from an equivalent lot purified from Cohn Fraction II+III silicon dioxide filter cake (FH006 fresh thaw, lane 4); and plasma-derived Factor H (CompTech, Tyler, Tex., lane 5), by SDS PAGE analysis of the reduced protein. Quantitation of the respective full length Factor H bands (e.g., the bands migrating at about 155 kDa) in FIG. 3 reveals that incubation of the purified Factor H composition results in further proteolytic clipping. For example, FH012 incubated at 4° C. for 13 days (lane 2) has nearly five times less fully intact Factor H (9% intact Factor H) than does freshly thawed FH012 (lane 3, 44% intact Factor H). Coupled with the concurrent increase in the intensity of the 120 kDa (corresponding to the larger proteolytic product) and 35 kDa (corresponding to the smaller proteolytic product) bands, these data evidence the presence of proteolytic enzymes in the purified Factor H composition.

The presence of amidolytic activity in Factor H lot FH012 was further confirmed by thromboelastographic (TEG) measurements showing that FH012 has a decreased clotting time as compared to normal citrated blood. Furthermore, western blotting analysis shows that Factor XI, an amidolytic component of the coagulation cascade, is present in Factor H lots FH006 and FH012 (FIG. 3B).

The proteolytic activity in Factor H lot FH012 was also observed in Cohn Fraction II+III silicon dioxide filter cake starting material. As shown in FIG. 4A, Factor H present in Cohn Fraction II+III silicon dioxide filter cake starting material was completely clipped to the 120 kDa fragment when the filter cake extract is incubated for 24 hours at 37° C. (lane 5). The proteolytic clipping was nearly eliminated by the addition of a protease inhibitor cocktail (Thermo Scientific, Rockford, Ill.), even after a 48 hour incubation at 37° C. (lane 10).

To demonstrate that the proteolytic activity present in the Fraction II+III silicon dioxide filter cake is responsible for proteolytic clipping of Factor H in the final composition, two Factor H purifications (FH091410 and FH100410) were performed as described in Example 1, except that protease inhibitors (Thermo Scientific cat: 78410) were added to the Fraction II+III extract of preparation FH100410 at a final concentration of 1%. As seen in FIG. 4B, the addition of protease inhibitors at the Fraction II+III extraction step significantly reduced clipping in the final Factor H composition from 52% to 15% (FIG. 4B, compare lane 4 with lane 3)

### Example 4

### Characterization of Factor H Proteolysis in Fractions Generated During Alcohol Fractionation of Human Plasma

The proteolytic state of Factor H in various plasma fractions produced during the manufacture of pooled human immunoglobulin G compositions, was investigated by western blot analysis. As seen in the western blot reproduced in FIG. 5A, the proteolysis of Factor H increases as Factor H progresses through the plasma fractionation process shown in FIG. 5B. For example, the percentage of clipped Factor H in the Fraction II+III precipitate is greater than in the Fraction I precipitate (e.g., as indicated by the ratio of the intensities of the 120 kDa and 155 kDa bands in lanes 8 and 6, respectively). These results suggest that Factor H compositions purified from Fraction I precipitates will have a better proteolytic profile (e.g., a smaller percentage of clipped Factor H) than Factor H compositions purified from Fraction II+III precipitates (e.g., from Cohn Fraction II+III silicon dioxide filter cake).

### Example 5

### Activity Profiles of Factor H Compositions Purified from Human Plasma

Functional effects of the observed proteolytic clipping of Factor H purified from Cohn Fraction II+III silicon dioxide filter cake were investigated by performing assays assessing various biological functions of Factor H. It was found that proteolytic clipping of Factor H did not have a significant effect on Factor I cofactor activity, but did significant reduce C3b binding, AH₅₀ heamolysis, and decay acceleration (DAF) activity in preparations isolated from Cohn Fraction II+III silicon dioxide filter cake.

FIG. 6 shows the results of Factor I cofactor assays performed with Factor H isolated from Cohn Fraction II+III silicon dioxide filter cake (FH002 and FH004) and plasma-derived Factor H (CompTech, Tyler, Tex.). These results demonstrate that proteolytic clipping of Factor H does not affect Factor I cofactor activity (compare FH002 and FH004 to CompTech).

FIG. 7 shows the results of decay acceleration (DAF) ELISA assays performed with Factor H isolated from Cohn Fraction II+III silicon dioxide filter cake (FH002, FH004, and FH070610) and a plasma-derived Factor H standard (CompTech, Tyler, Tex.).

FIG. 8 shows the results of AHSO heamolysis activity assays performed with Factor H isolated from Cohn Fraction II+III silicon dioxide filter cake (FH006) and plasma-derived Factor H (CompTech, Tyler, Tex.). These results demonstrate that the proteolytic clipping in Factor H observed in compositions prepared from reduces Cohn Fraction II+III silicon dioxide filter cake reduces the compositions ability to promote haemolysis.

Taken together, these results suggest that proteolytically clipped Factor H has a reduced binding affinity for surface bound C3b. Without being bound by theory, the reduced binding affinity is likely due to a disruption in the C3b binding site found in short consensus repeat (SCR) domains 6-8 adjacent to the proteolytically clipped site in SCR domain 5. The finding that Factor I cofactor activity is not disrupted in Factor H preparations from Cohn Fraction II+III silicon dioxide filter cake is consistent with clipping in SCR domain 5, because SCR domains 1-4, critical for Factor I cofactor activity, remain intact.

### Example 6

### Characterization of Factor H Stability in Fraction I Precipitate Suspensions

As demonstrated in the examples above, Cohn Fraction II+III precipitates and derivatives thereof (e.g., Cohn Fraction II+III silicon dioxide filter cake) contain amidolytic activities that proteolytically clip Factor H. This clipped Factor H has reduced biological activity, as demonstrated in C3b binding, AHSO heamolysis, and decay acceleration (DAF) activity assays. Because plasma fractions formed upstream of Fraction II+III precipitation contained lower levels of Factor H proteolysis (compare lanes 4-6 to lane 8 in FIG. 5A), Fraction I precipitate was investigated as a potential source material for the isolation of Factor H.

The stability of Factor H in Cohn Fraction I precipitate was examined as described for Fraction II+III silicon dioxide filter cake in Example 3. Briefly, Fraction I precipitate was suspended in buffer and incubated at 37° C. for 1 hour with and without addition of the protease inhibitor Aprotinin. Proteolytic clipping upon incubation was then assessed by western blotting SDS-PAGE analysis. As shown in FIG. 9, Factor H present in suspended Fraction I precipitate is not further proteolyzed upon incubation at 37° C. in the absence of the protease inhibitor (compare lanes 5 and 8). This demonstrates that proteolytic enzymes found to clip Factor H in Fraction II+III precipitates, including Cohn Fraction II+III silicon dioxide filter cake, are not present in Fraction I precipitates.

### Example 7

### PEG Precipitation Enrichment of Factor H from Cohn Fraction I Precipitate

As outlined above, previous studies described in U.S. Pat. No. 8,304,524 suggest that enrichment of Factor H from Fraction II+III filter cake extracts is achieved by performing a first precipitation at 15% ethanol, to remove contaminants, and a second precipitation at 25% ethanol, to recover Factor H. This strategy is likely effective, without being bound by theory, because Fraction II+III precipitates are prepared using about 25% ethanol. Thus some impurities can be precipitated out by using a lightly lower ethanol concentration (e.g., 15% ethanol). However, because the Fraction I precipitate is prepared using only 8% ethanol, initial enrichment performed with 15% ethanol would not provide separation. Furthermore, the low ethanol concentration (8%) provides a limited, if any, window to perform partial precipitations.

To search for other strategies of enriching Factor H from Fraction I precipitate, mild PEG precipitation conditions were evaluated. Advantageously, it was found that at neutral to slightly basic pH, major impurities of the suspended Fraction I (e.g., fibrinogen) were precipitated at very low concentrations of PEG, which are insufficient to precipitate Factor H. For example, FIGS. 14 and 15 show SDS-PAGE and Western blot analysis of PEG precipitation studies on suspended Fraction I precipitates performed at pH 8.0 with increasing PEG concentrations from 1% PEG 4 k to 6% PEG 4 k, as indicated. Strikingly, as shown in FIG. 14B, a large fraction of lower molecular weight impurities (e.g., 50-65 kD; presumably fibrinogen) are precipitated out of the suspension at PEG concentrations as low as 2% PEG 4 k (see, FIG. 14B, lane 4). In contrast, as seen in the anti-Factor H western blots reproduced in FIG. 15, only a small fraction of Factor H is precipitated out of the Fraction I suspension at PEG concentrations below 6%. Even at 6%, it appears that less than half of the Factor H content is precipitated. Thus, use of PEG concentrations between about 2% and about 6% will provide substantial enrichment of Factor H in the Fraction I suspension.

### Example 8

### Purification of Factor H from Cohn Fraction I Precipitate

This example demonstrates that Factor H compositions with 10% or less Factor H proteolytic clipping can be purified from Cohn Fraction I precipitate generated as a byproduct of the manufacturing process for pooled human IgG products.

Briefly, frozen Fraction I precipitate prepared under standard conditions (e.g., incubation of cryo-poor pooled plasma at −1° C. (pH 7.2±0.2) after addition of denatured ethanol to a final concentration 8% (v/v)) was thawed in an extraction buffer containing 25 mM Tris, 200 mM NaCl, 5 mM EDTA (pH 8). The solution was stirred at room temperature and the Fraction I precipitate was manually mashed until suspended. Typically, some insoluble material remains in the extract.

Polyethylene glycol (PEG) 4 k was slowly added to the suspension to a final concentration of 5% (w/v) and the mixture was stirred for at least 30 min, to precipitate contaminants. After incubation, the PEG suspension was centrifuged at 4° C. for 20 minutes at 15,000×g to pellet precipitated material. PEG 4 k was added to the centrifuged supernatant to a final concentration of 12% (w/v) and the mixture was stirred for at least 30 minutes at 4° C., to precipitate Factor H. The 12% PEG suspension was centrifuged at 4° C. for 20 minutes at 15 k×g to pellet precipitated material. The precipitate, containing Factor H, was then suspended in buffer containing 25 mM Tris, 50 mM NaCl, 5 mM EDTA (pH 8). Although precipitation was used to separate PEG precipitated material from the supernatant, any means known in the art for separating protein precipitates may be used (e.g., filtration).

Factor H in the second PEG precipitate suspension was further enriched by anion exchange chromatography. Briefly, suspended 12% PEG precipitate was loaded onto DEAE Sepharose resin equilibrated with buffer containing 65 mM sodium chloride, washed using the same buffer, and then eluted with buffer containing 120 mM sodium chloride, according to the detailed parameters outlined in Table 3. DEAE enrichment was analyzed by SDS-PAGE (FIG. 10A) and western blot (FIG. 10B) analyses. Factor H containing fractions from the DEAE elution were pooled and diluted with buffer containing 25 mM Tris, 5 mM EDTA (pH 8.0) to a final salt concentration of 50 mM.

TABLE 3
Parameters used for DEAE Sepharose chromatography of
Factor H from 12% PEG precipitate.

| Parameter: | Value: |
|---|---|
| Media | GE DEAE Sepharose FF |
| Column Body | XK 50/30 |
| Bed Height | 22 cm |
| Column Volume | 432 mL |
| Flow Rate | 50 mL/min |

| Pump Inlet: | Buffer: |
|---|---|
| A1 | 25 mM Tris, 5 mM EDTA (pH 8) |
| A2 | H₂O |
| B1 | 25 mM Tris, 1M NaCl, 5 mM EDTA (pH 8) |
| B2 | H₂O |

Method

| | |
|---|---|
| Equilibrate | 5 CV @ 6.5% B1 |
| Load | Factor H Extract (25 mL/min load) |
| Wash | 5 CV @ 6.5% B1 |
| Elute | 5 CV @ 12% B1 (Fractions Collected) |
| Regenerate | 5 CV @ 100% B1 |
| Wash | 5 CV @ 50% A2/B2 |

Factor H in the DEAE eluate was further enriched by heparin affinity chromatography. Briefly, the diluted DEAE eluate was loaded onto heparin Sepharose resin equilibrated with buffer containing 50 mM sodium chloride, washed using the same buffer, and then eluted with buffer containing 150 mM sodium chloride, according to the detailed parameters outlined in Table 4. Factor H containing fractions from the heparin affinity elution were pooled and diluted with buffer containing 25 mM Tris, 5 mM EDTA (pH 8.0) to a final salt concentration of 50 mM.

TABLE 4
Parameters used for heparin affinity chromatography of
Factor H from DEAE eluate.

| Parameter: | Value: |
|---|---|
| Media | GE Heparin Sepharose FF |
| Column Body | XK 50/20 |
| Bed Height | 10.5 cm |
| Column Volume | 206 mL |
| Flow Rate | 40 mL/min |

| Pump Inlet: | Buffer: |
|---|---|
| A1 | 25 mM Tris, 5 mM EDTA (pH 8) |
| A2 | H₂O |
| B1 | 25 mM Tris, 1M NaCl, 5 mM EDTA (pH 8) |
| B2 | H₂O |

Method

| | |
|---|---|
| Equilibrate | 5 CV @ 5% B1 |
| Load | Diluted DEAE Fraction Pool (30 mL/min load) |
| Wash | 5 CV @ 5% B1 |
| Elute | 5 CV @ 15% B1 (Fractions Collected) |
| Regenerate | 5 CV @ 100% B1 |
| Wash | 5 CV @ 50% A2/B2 |

Factor H in the heparin affinity eluate was further enriched by anion exchange chromatography. Briefly, the diluted heparin affinity eluate was loaded onto Q Sepharose resin equilibrated with buffer containing 50 mM sodium chloride, washed using the same buffer, and then eluted stepwise with buffers containing 100 mM sodium chloride, 150 mM sodium chloride, and 300 mM sodium chloride, according to the detailed parameters outlined in Table 5. Factor H containing fractions from the Q elution were pooled and concentrated into PBS buffer using Amicon stirred cells having 30 kDa or 50 kDa MWCO PES membranes (Millipore or Pall) under nitrogen gas at 55-60 psi.

TABLE 5
Parameters used for Q Sepharose anion exchange
chromatography of Factor H from heparin affinity eluate.

| Parameter: | Value: |
|---|---|
| Media | GE Q Sepharose FF |
| Column Body | XK 16/20 |
| Bed Height | 10.5 cm |
| Column Volume | 21 mL |
| Flow Rate | 10 mL/min |

| Pump Inlet: | Buffer: |
|---|---|
| A1 | 25 mM Tris (pH 8.0), 5 mM EDTA |
| A2 | H₂O |
| B1 | 25 mM Tris (pH 8.0), 1M NaCl, 5 mM EDTA |
| B2 | H₂O |

Method

| | |
|---|---|
| Equilibrate | 5 CV @ 5% B1 |
| Load | Diluted Heparin Fraction Pool |
| Wash | 5 CV @ 5% B1 |
| Elute (low salt) | 5 CV @ 10% B1 (Fractions Collected) |
| Elute (high salt) | 5 CV @ 15% B1 (Fractions Collected) |
| Wash | 5 CV @ 30% B1 (Fractions Collected) |
| Regenerate | 5 CV @ 100% B1 |
| Wash | 5 CV @ 50% A2/B2 |

Heparin affinity and Q Sepharose enrichment of Factor H was analyzed by SDS-PAGE (FIG. 11A) and western blot (FIG. 11B) analyses. Factor H eluted from the Q Sepharose resin with 150 mM sodium chloride was largely intact (>90% full length), comparable to plasma-derived Factor H (CompTech, Tyler, Tex.; lanes 9 and 1 in FIG. 11B, respectively).

### Example 9

### Purification and Analysis of Factor H Purified from Cohn Fraction I Precipitate

Factor H was purified from 1 kg of Cohn Fraction I precipitate, further evidencing the feasibility of isolating Factor H in large scale from Fraction I precipitate, which is normally discarded during manufacturing of pooled human IgG compositions. Analysis of the final composition demonstrates Factor H activity, purity, and intactness comparable to plasma-derived preparations.

Briefly, 1 kg of Fraction I precipitate, prepared as in Example 8, was thawed in buffer containing 25 mM Tris (pH 8.0), 200 mM NaCl, and 5 mM EDTA to yield 3 L of final extract. The solution was stirred at room temperature and the Fraction I precipitate paste was manually mashed until suspended. Some insoluble material remained in the extract. The suspension was centrifuged at 4° C. for 20 min at 20,000×g to remove the insoluble material.

Polyethylene glycol (PEG) 4 k was slowly added to the suspension to a final concentration of 4% (w/v) and the mixture was stirred for at least 30 min, to precipitate contaminants. After incubation, the PEG suspension was centrifuged at 4° C. for 20 minutes at 20,000×g to pellet precipitated material. PEG 4 k was added to the centrifuged supernatant to a final concentration of 12% (w/v) and the mixture was stirred for at least 30 minutes at 4° C., to precipitate Factor H. The 12% PEG suspension was centrifuged at 4° C. for 20 minutes at 20,000×g to pellet precipitated material. The precipitate, containing Factor H, was then suspended in buffer containing 25 mM Tris (pH 8.0), 50 mM NaCl, and 5 mM EDTA. The final suspension was filtered through a 0.2 μm filter and stored at 4° C. Although precipitation was used to separate PEG precipitated material from the supernatant, any means known in the art for separating protein precipitates may be used (e.g., filtration).

Factor H was enriched from the suspended PEG precipitate by sequential DEAE Sepharose anion exchange, heparin affinity, and Q Sepharose anion exchange chromatography steps as described in Example 8. The Factor H containing Q Sepharose eluate was concentrated in Amicon stirred cells with 50 kDa MWCO PES membranes under nitrogen gas at 55 psi, to a final concentration of 30-50 mg/mL in PBS. The concentrated Factor H was then applied to a Superdex 200 column for final polishing. The Factor H containing SEC fractions were concentrated in an Amicon cell as above, filtered through 0.2 μm Mustang E sterile filter units (Pall Corporation, Port Wash., NY) for terminal filtration and endotoxin removal, prior to freezing aliquots in liquid nitrogen for storage at −70° C.

Unlike Factor H compositions purified from Cohn Fraction II+III silicon dioxide filter cake, the final Factor H composition isolated from the Fraction I precipitate was largely intact (92%), comparable to plasma-derived Factor H preparations (CompTech, Tyler, Tex.). Results of biochemical analysis of the final product are shown in Table 6.

TABLE 6
Biochemical analysis of Factor H composition purified from
Cohn Fraction I precipitate.

| Assay | Result |
|---|---|
| Protein Concentration | 16.7 mg/mL |
| (BCA) | |
| Purity | 99.6% |
| (SEC-HPLC) | |
| Purity | 100% (No visible impurities with 2 μg load |
| (non-reduced SDS PAGE) | stained with Coomassie) |
| Intactness | 92% intact |
| (reduced SDS PAGE) | 8% proteolytically clipped |
| C3b binding | 0.99 (log ratio to CompTech standard; run 1) |
| ELISA | 1.12 (log ratio to CompTech standard; run 2) |
| PKA-coagulation | <3.25% (1.6 IU/mL) |
| Amidolytic Activity | None Detected |
| (S-2302 broad spectrum) | |
| TEG-coagulation | Normal |
| Endotoxin-LAL | 0.35 EU/mL |

### Example 10

### Biochemical Analysis of Factor H Purified from Fraction I Precipitate and Comparison to Factor H Purified from Fraction II+III silicon Dioxide Filter Cake

As discussed in Example 3, Factor H compositions prepared from Fraction II+III silicon dioxide filter cake contain proteases that proteolytically clip Factor H at SCR domain 5. The stability of Factor H purified from Fraction I precipitate, prepared as described in Example 9, was analyzed in the same fashion. Briefly, SDS-PAGE analysis of Factor H lot pFH110411 (FIG. 12) revealed that Factor H is stable when incubated at 4° C. for two weeks in the absence of protease inhibitors, demonstrating substantially lower levels of proteolytic activity in these preparations, as compared to Factor H composition isolated from Fraction II+III silicon dioxide filter cake. Furthermore, the proteolytic profile of the Fraction I precipitate-derived Factor H incubated for two weeks is nearly identical to that of commercially available plasma-derived Factor H (CompTech, Tyler, Tex.).

Furthermore, as discussed in Example 5, Factor H prepared from Fraction II+III silicon dioxide filter cake has reduced binding to C3b in ELISA assays. The C3b binding activity of Factor H purified from Fraction I precipitate (189-2; FH037), as described in Example 9, was compared to C3b binding activities of Factor H purified from Fraction II+III silicon dioxide filter cake (FH12), commercially available plasma-derived Factor H(CT FH; CompTech, Tyler, Tex.), and recombinant Factor H prepared in-house at Baxter (rFHlot5). The results of this comparison, shown in FIG. 13, demonstrate that Factor H purified from Fraction I precipitate binds better to immobilized (e.g., surface bound) C3b than does proteolytically clipped Factor H isolated from Fraction II+III silicon dioxide filter cake, and comparably to commercially available plasma-derived Factor H.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method for preparing an enriched Factor H composition from plasma, the method comprising the steps:
(A) precipitating Factor H from a Cohn plasma pool, in a first precipitation step, the first precipitation step performed at a final concentration of from 6% to 10% alcohol at a pH of from 7.0 to 7.5, thereby forming a first precipitate and a first supernatant;
(B) extracting the Factor H from the first precipitate with a first Factor H extraction buffer, thereby preparing an extracted Factor H composition;
(C) admixing polyethylene glycol (PEG) into an extracted Factor H composition, in a second precipitation step, the second precipitation step performed at a final concentration of from 2% to 7% PEG at a pH of from 7.0 to 9.0, thereby forming a second precipitate and a second supernatant;
(D) admixing PEG into the second supernatant, in a third precipitation step, the third precipitation step performed at a final concentration of from 10% to 20% PEG at a pH of from 7.0 to 9.0, thereby forming a third precipitate and a third supernatant; and
(E) extracting the Factor H from the third precipitate with a second Factor H extraction buffer, thereby forming an enriched Factor H composition.

2. The method of claim 1, wherein the Cohn plasma pool comprises cryo-poor plasma.

3. The method of claim 1, wherein the final concentration of alcohol in the first precipitation step is 8±1%.

4. The method of claim 1, wherein the pH of the first precipitation step is 7.2±0.4.

5. The method of claim 1, wherein the first Factor H extraction buffer has a pH of from 7.0 to 9.0.

6. The method of claim 1, wherein the first Factor H extraction buffer has a pH of 8.0±0.5.

7. The method of claim 1, wherein the first Factor H extraction buffer has a conductivity of from 7 mS/cm to 32 mS/cm.

8. The method of claim 1, wherein the first Factor H extraction buffer has a conductivity of from 11 mS/cm to 22 mS/cm.

9. The method of claim 1, wherein the final concentration of PEG in the second precipitation step is 2% to 5%.

10. The method of claim 1, wherein the final concentration of PEG in the second precipitation step is 4±1%.

11. The method of claim 1, wherein the pH of the second precipitation step is 8±0.5.

12. The method of claim 1, wherein the final concentration of PEG in the third precipitation step is from 10% to 15%.

13. The method of claim 1, wherein the final concentration of PEG in the third precipitation step is 12±1%.

14. The method of claim 1, wherein the pH of the second precipitation step is 8±0.5.

15. The method of claim 1, wherein the second Factor H extraction buffer has a pH of from 7.0 to 9.0.

16. The method of claim 1, wherein the second Factor H extraction buffer has a pH of 8.0±0.5.

17. The method of claim 1, wherein the second Factor H extraction buffer has a conductivity of from 2 mS/cm to 10 mS/cm.

18. The method of claim 1, wherein the second Factor H extraction buffer has a conductivity of from 5 mS/cm to 9 mS/cm.

19. The method of claim 1, wherein the alcohol is ethanol.

20. The method of claim 1, further comprising at least one further enrichment step.

21. The method of claim 1, further comprising at least one anion exchange chromatography enrichment step.

22. The method of claim 21, wherein the anion exchange chromatography enrichment step includes binding Factor H to a diethylaminoethyl (DEAE) chromatography material.

23. The method of claim 1, further comprising at least one heparin affinity chromatography enrichment step.

24. The method of claim 1, further comprising at least one size exclusion chromatography enrichment step.

25. The method of claim 1, further comprising at least one viral inactivation or removal step.
